SEQUENCE LISTING (78 sequences)

SEQ ID NO: 1 - DNA, 13011 bp, Artificial Sequence
  Synthetic VSV-DELTA-G-LASV; Wild-type VSV containing a codon-optimized Lassa glycoprotein in place of the VSV glycoprotein. `
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaactaacagagatcgatctgtttccttgacactatgggtcagatcgttactttcttccaggaagtaccccatgttatagaagaggttatgaatatagtccttattgctttgagcgtcttggcggtgctgaaaggtctctacaactttgcaacttgtggtctggtcggcctggtgactttcctcctgctgtgtggccggtcctgcacgactagcttgtacaaaggggtctatgagttgcagactctcgagcttaatatggaaacattgaacatgacaatgccgctctcatgcacaaagaacaactcccaccactatataatggtgggaaatgagactgggttggaacttacgctgaccaacactagtattattaaccataagttctgtaacctctctgatgcccacaagaaaaatctctatgatcatgcgttgatgtcaataataagcacatttcatctgtctatacctaattttaaccaatatgaagctatgtcttgtgatttcaacgggggtaagatatccgtccaatataatttgtctcactcctacgctggagatgctgcaaaccattgcggcactgttgccaacggagttctgcaaaccttcatgcggatggcatggggtggtagttacatcgccttggattctgggagaggcaattgggactgcattatgacttcctaccagtacctcattattcaaaatacgacgtgggaggatcattgccaattttctaggcccagcccgattgggtacctcggactcctttcccaacggactagggatatatacattagtcggaggctgcttgggaccttcacatggacactctctgactcagaaggcaaggatacgcccggtggatactgtttgaccagatggatgcttattgaggccgagcttaaatgctttggtaataccgccgtagcgaagtgtaatgaaaaacacgacgaggaattctgcgacatgctcaggttgttcgattttaacaaacaggctatacaacgcctgaaagcagaggcccaaatgtctatccaactcatcaacaaggcagtcaatgcactcattaatgatcagctgattatgaagaaccacctgagggatataatgggcattccttactgcaactattctaagtattggtatctgaaccatacaactacgggacggacgtccctgcctaaatgttggcttgtcagtaatggatcctacctcaatgagacacacttctctgacgacatagaacaacaggcggataatatgattactgagatgttgcagaaagagtatatggagagacagggcaaaacaccgcttggcctcgtggacctgtttgtcttctctacttccttctatctcatttctatctttcttcatcttgttaagatcccgacccacaggcatatagtgggcaagagttgtccaaagccccaccggctgaatcatatgggcatttgcagttgtggtctgtacaaacagcccggagtgcccgtcaagtggaaaaggtgactcaaatcctgcacaacagattcttcatgtttggaccaaatcaacttgtgataccatgctcaaagaggcctcaattatatttgagtttttaatttttatgaaaaaaactaacagcaatcatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggctcgggcggtggtgggtcgggtggcggcggatctatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaactcaaatcctgcacaacagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 2 - DNA, 1476 bp, Lassa virus
  MISC_FEATURE (1)..(1476): Lassa Glycoprotein Josiah (Non codon-optimized)
atgggacaaatagtgacattcttccaggaagtgcctcatgtaatagaagaggtgatgaacattgttctcattgcactgtctgtactagcagtgctgaaaggtctgtacaattttgcaacgtgtggccttgttggtttggtcactttcctcctgttgtgtggtaggtcttgcacaaccagtctttataaaggggtttatgagcttcagactctggaactaaacatggagacactcaatatgaccatgcctctctcctgcacaaagaacaacagtcatcattatataatggtgggcaatgagacaggactagaactgaccttgaccaacacgagcattattaatcacaaattttgcaatctgtctgatgcccacaaaaagaacctctatgaccacgctcttatgagcataatctcaactttccacttgtccatccccaacttcaatcagtatgaggcaatgagctgcgattttaatgggggaaagattagtgtgcagtacaacctgagtcacagctatgctggggatgcagccaaccattgtggtactgttgcaaatggtgtgttacagacttttatgaggatggcttggggtgggagctacattgctcttgactcaggccgtggcaactgggactgtattatgactagttatcaatatctgataatccaaaatacaacctgggaagatcactgccaattctcgagaccatctcccatcggttatctcgggctcctctcacaaaggactagagatatttatattagtagaagattgctaggcacattcacatggacactgtcagattctgaaggtaaagacacaccagggggatattgtctgaccaggtggatgctaattgaggctgaactaaaatgcttcgggaacacagctgtggcaaaatgtaatgagaagcatgatgaggaattttgtgacatgctgaggctgtttgacttcaacaaacaagccattcaaaggttgaaagctgaagcacaaatgagcattcagttgatcaacaaagcagtaaatgctttgataaatgaccaacttataatgaagaaccatctacgggacatcatgggaattccatactgtaattacagcaagtattggtacctcaaccacacaactactgggagaacatcactgcccaaatgttggcttgtatcaaatggttcatacttgaacgagacccacttttctgatgatattgaacaacaagctgacaatatgatcactgagatgttacagaaggagtatatggagaggcaggggaagacaccattgggtctagttgacctctttgtgttcagtacaagtttctatcttattagcatcttccttcacctagtcaaaataccaactcataggcatattgtaggcaagtcgtgtcccaaacctcacagattgaatcatatgggcatttgttcctgtggactctacaaacagcctggtgtgcctgtgaaatggaagagatga

SEQ ID NO: 3 - DNA, 1476 bp, Lassa virus
  MISC_FEATURE (1)..(1476): Lassa Glycoprotein Josiah (Human Codon- optimized)
atgggtcagatcgttactttcttccaggaagtaccccatgttatagaagaggttatgaatatagtccttattgctttgagcgtcttggcggtgctgaaaggtctctacaactttgcaacttgtggtctggtcggcctggtgactttcctcctgctgtgtggccggtcctgcacgactagcttgtacaaaggggtctatgagttgcagactctcgagcttaatatggaaacattgaacatgacaatgccgctctcatgcacaaagaacaactcccaccactatataatggtgggaaatgagactgggttggaacttacgctgaccaacactagtattattaaccataagttctgtaacctctctgatgcccacaagaaaaatctctatgatcatgcgttgatgtcaataataagcacatttcatctgtctatacctaattttaaccaatatgaagctatgtcttgtgatttcaacgggggtaagatatccgtccaatataatttgtctcactcctacgctggagatgctgcaaaccattgcggcactgttgccaacggagttctgcaaaccttcatgcggatggcatggggtggtagttacatcgccttggattctgggagaggcaattgggactgcattatgacttcctaccagtacctcattattcaaaatacgacgtgggaggatcattgccaattttctaggcccagcccgattgggtacctcggactcctttcccaacggactagggatatatacattagtcggaggctgcttgggaccttcacatggacactctctgactcagaaggcaaggatacgcccggtggatactgtttgaccagatggatgcttattgaggccgagcttaaatgctttggtaataccgccgtagcgaagtgtaatgaaaaacacgacgaggaattctgcgacatgctcaggttgttcgattttaacaaacaggctatacaacgcctgaaagcagaggcccaaatgtctatccaactcatcaacaaggcagtcaatgcactcattaatgatcagctgattatgaagaaccacctgagggatataatgggcattccttactgcaactattctaagtattggtatctgaaccatacaactacgggacggacgtccctgcctaaatgttggcttgtcagtaatggatcctacctcaatgagacacacttctctgacgacatagaacaacaggcggataatatgattactgagatgttgcagaaagagtatatggagagacagggcaaaacaccgcttggcctcgtggacctgtttgtcttctctacttccttctatctcatttctatctttcttcatcttgttaagatcccgacccacaggcatatagtgggcaagagttgtccaaagccccaccggctgaatcatatgggcatttgcagttgtggtctgtacaaacagcccggagtgcccgtcaagtggaaaaggtga

SEQ ID NO: 4 - protein, 491 aa, Lassa virus
  MISC_FEATURE (1)..(491): Amino acid
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 5 - DNA, 2031 bp, Ebola virus
  MISC_FEATURE (1)..(2031): Ebola Glycoprotein Wild-type (Not codon- optimized)
atgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

SEQ ID NO: 6 - DNA, 2031 bp, Ebola virus
  MISC_FEATURE (1)..(2031): Codon-Optimized
atgggtgtcacgggaattctccaacttccgagagaccgatttaaacgcacatcattctttctttgggttattattctttttcaacgaacatttagcatacctctcggcgtcatacataattctactctccaggtgtcagatgttgataagctggtgtgtagggacaaactctcatctactaatcaattgaggagcgtgggattgaacttggagggaaacggggtggctaccgacgtcccgagcgccacgaagaggtggggatttcgatctggtgttcctccgaaagtggtaaattacgaggccggcgagtgggccgaaaattgttacaacttggagatcaaaaaaccagatggaagtgagtgtttgccagcggcgccggacggcataagagggttcccgaggtgtcgatatgttcacaaagtatcagggacgggtccatgcgcgggagacttcgctttccataaagagggagctttcttcttgtatgatagactcgcatccacggtgatttatagaggcaccacttttgccgaaggtgtcgtcgcctttttgatactgccccaagcgaagaaagatttctttagcagccatccactcagggagcctgtaaatgcaacagaggatccctcttctggttactacagtacaacgattaggtaccaggcaaccggtttcggcacgaacgaaactgagtatctgtttgaagtggacaatctgacatatgtgcaactcgaatcaagattcaccccgcagttcctgctgcaacttaacgaaactatttacaccagtgggaagcgatctaataccactggcaaactcatatggaaggtaaatcccgagatcgataccaccattggcgagtgggcgttctgggagacaaaaaaaaatctcaccagaaaaatcaggagcgaggaactgagtttcactgtcgtatccaatggagctaaaaacattagtggccaatctccagcgcgcacttcatcagatccaggtacgaatacaaccactgaggatcataaaatcatggcttctgagaattctagtgccatggtacaagtgcactcccaagggagggaagccgctgtgtctcatttgaccactctcgctactatttctacctccccgcaaagtctgactaccaagccggggccggataactctactcacaacacccccgtatacaagctggatattagcgaggccacccaagtcgagcagcatcatagacgcacggacaatgacagtactgcttcagataccccaagtgctacgactgctgcagggccgccgaaggctgagaacaccaatacctccaagtctacggattttctcgatccggctacaactacttctccgcaaaatcattccgaaacggcgggtaataataacacacatcaccaggacacaggtgaggagtccgcttctagcggaaagctcggtctgatcaccaacacaatcgcgggagtagcgggactcattacaggtggtcggaggactaggcgggaggccattgtcaacgcacagcctaagtgtaatcctaatcttcattattggacgactcaggatgagggagcagccattggtttggcttggatcccttatttcggtcccgctgcggaaggaatatatattgaagggcttatgcacaaccaggatggactgatatgtggtttgcgacagcttgcgaatgaaacgacgcaggcattgcaattgttccttagagccacgacggaactccgaaccttttccattctcaatcgcaaggccattgacttcttgttgcaacgctggggcggaacctgtcacatattgggaccggattgctgcatcgaaccgcacgattggaccaagaacattaccgacaaaattgaccaaattatacacgacttcgttgataagacactcccggatcaaggcgacaacgataactggtggaccggctggcggcaatggatccctgctggtataggcgtaacgggtgtcattatagcagtcatcgcacttttttgcatctgtaaattcgtgttctag

SEQ ID NO: 7 - protein, 676 aa, Ebola virus
  MISC_FEATURE (1)..(676): Amino acid sequence
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 8 - DNA, 2946 bp, Chikungunya virus
  MISC_FEATURE (1)..(2946): Chikungunya Glycoprotein Wild-Type (Not Codon- Optimized)
atgtgcctgttggcaaataccacgttcccctgctcccagcccccttgcataccctgctgctacgaaaaggaaccggaggaaaccctacgcatgcttgaggacaacgtcatgagacctgggtactatcagctgctacaagcatcattaacatgttctccccaccgccagcgacgcagcaccaaggacaacttcaatgtctataaagccacaagaccatacctagctcactgtcccgactgtggagaagggcactcgtgccatagtcccgtagcactagaacgcatcagaaatgaagcgacagacgggacgctgaaaatccaggtctccttgcaaattggaatagggacggatgatagccatgattggaccaagctgcgttacatggacaatcacataccagcagacgcagggagggccgggctatttgtaagaacatcagcaccatgcacgattactggaacaatgggacacttcatcctggcccgatgtccgaaaggagaaactctgacggtgggattcactgacagtaggaagattagtcactcatgtacgcacccatttcaccacgaccctcctgtgataggccgggaaaaattccattcccgaccgcagcacggtaaagagctaccttgcagcacgtacgtgcagagcaacgccgcaactgccgaggagatagaggtacacatgcccccagacacccctgatcgcacattgctgtcacaacagtccggcaacgtaaagatcacagtcaatagtcagacggtgcggtataagtgtaattgcggtggctcaaatgaaggactaataactacagataaagtgattaataactgcaaggttgatcaatgtcatgccgcggtcaccaatcacaaaaagtggcagtataactcccctctggtcccgcgtaacgctgaactcggggaccgaaaaggaaaaattcacatcccgtttccgctggcaaatgtaacatgcatggtgcctaaagcaaggaaccccaccgtgacgtacgggaaaaaccaagtcatcatgctactgtatcctgaccacccaacactcctgtcctaccggagtatgggagaagaaccaaactatcaagaagagtgggtgacgcacaagaaggaggtcgtgctaaccgtgccgactgaagggctcgaggttacgtggggcaacaacgagccgtataagtattggccgcagttatctgcaaacggtacagcccacggccacccgcatgagataatcttgtactattatgagctgtaccctactatgactgtagtagttgtgtcagtggcctcgttcatactcctgtcgatggtgggtatggcagtggggatgtgcatgtgtgcacgacgcagatgcatcacaccatacgaactgacaccaggagctaccgtccctttcctgcttagcctaatatgctgcatcagaacagctaaagcggccacataccaagaggctgcggtatacctgtggaacgagcagcaacctttgttttggctacaagcccttattccgctggcagccctgattgtcctatgcaactgtctgagactcttaccatgctgttgtaaaacgttggcttttttagccgtaatgagcatcggtgcccacactgtgagcgcgtacgaacacgtaacagtgatcccgaacacggtgggagtaccgtataagactctagtcaacagaccgggctacagccccatggtactggagatggagctactgtcagtcactttggagccaacgctatcgcttgattacatcacgtgcgaatacaaaaccgtcatcccgtctccgtacgtgaaatgctgcggtacagcagagtgcaaggacaaaaacctacctgactacagctgtaaggtcttcaccggcgtctacccatttatgtggggcggcgcctactgcttctgcgacgctgaaaacacgcaattgagcgaagcacatgtggagaagtccgaatcatgcaaaacagaatttgcatcagcatacagggctcataccgcatccgcatcagctaagctccgcgtcctttaccaaggaaataacatcactgtaactgcctatgcaaacggcgaccatgccgtcacagttaaggacgccaaattcattgtggggccaatgtcttcagcctggacaccttttgacaacaaaatcgtggtgtacaaaggtgacgtttacaacatggactacccgccctttggcgcaggaagaccaggacaatttggcgatatccaaagtcgcacgcctgagagcaaagacgtctatgctaacacacaactggtactgcagagaccggctgcgggtacggtacacgtgccatactctcaggcaccatctggctttaagtattggttaaaagaacgaggggcgtcgctacagcacacagcaccatttggctgccaaatagcaacaaacccggtaagagcgatgaactgcgccgtagggaacatgcccatctccatcgacataccggatgcggccttcactagggtcgtcgacgcgccctctttaacggacatgtcatgcgaggtaccagcctgcacccattcctcagactttgggggcgtcgccattattaaatatgcagtcagcaagaaaggcaagtgtgcggtgcattcgatgaccaacgccgtcactatccgggaagctgagatagaagttgaagggaattctcagctgcaaatctctttctcgacggccttggccagcgccgaattccgcgtacaagtctgttctacacaagtacactgtgcagccgagtgccaccctccgaaggaccacatagtcaactacccggcgtcacataccaccctcggggtccaggacatttccgctacggcgatgtcatgggtgcagaagatcacgggaggtgtgggactggttgtcgctgttgcagcactgattctaatcgtggtgctatgcgtgtcgttcagcaggcactaa

SEQ ID NO: 9 - DNA, 2946 bp, Chikungunya virus
  MISC_FEATURE (1)..(2946): >CHIK_ E3-E2-6K-E1_Codon-Optimized
atgtgtcttctggctaacacgaccttcccctgtagccagccgccctgcataccctgttgctacgagaaagaaccagaggagacgctgcgaatgcttgaagacaacgtgatgcgaccggggtactaccaattgctccaggcctcactcacctgtagcccacacaggcagagaagatctaccaaggataattttaatgtttacaaagcaacgaggccctacctggcgcattgccccgactgcggcgaggggcactcctgccactcccccgtggcccttgagcgcattaggaatgaggcgactgacggaaccctcaaaattcaagtttcactccaaataggtatcggtactgacgattcccatgattggacaaagcttcggtatatggataatcacatccccgcagacgcgggcagagctgggttgttcgttcgaacaagcgcgccctgtacgataacgggtaccatgggtcattttattctggccagatgccccaagggtgaaacactgaccgtcggattcacggatagccgcaaaatctcacattcctgtactcatcctttccatcacgacccgcctgtcatcggacgggagaagtttcattcccggccccaacatgggaaagagctgccgtgctccacgtatgttcaaagcaacgctgcaactgcggaagaaatcgaagtacacatgcctccggacacgcctgacaggacactgctgagccaacagtcaggaaacgttaaaattactgtaaactcacaaaccgtgcggtacaagtgtaattgtggagggagtaacgaagggctcattacaacagacaaggtcataaacaactgtaaagttgaccagtgtcatgcggccgtgacgaaccataaaaagtggcaatacaactctccgctcgtgcccaggaacgctgagctcggagatagaaaaggaaaaatccatatcccttttccccttgccaatgttacgtgtatggtgccaaaagcgcggaatcccaccgttacttatggaaagaaccaagttattatgcttctctatcccgatcacccgacgctgctgtcctacaggtctatgggtgaggaaccaaattatcaagaggaatgggtgacacacaagaaggaggtcgtgcttaccgtcccgacggaggggcttgaagttacctggggaaacaacgaaccatataagtactggccccaactgagtgcaaacggtacggctcacggtcaccctcacgagatcatactctactactatgaactgtaccccaccatgaccgtggtagttgtatccgtcgcgagcttcatactcctttcaatggtcgggatggcagtaggcatgtgtatgtgcgcacgaagaaggtgtataacgccttatgaattgactcctggtgcaaccgttccttttctgctgtcactcatttgttgtatcaggaccgcaaaggctgctacttaccaggaggctgctgtttacctgtggaatgagcaacagcccctgttttggttgcaggctctgatcccactcgcagccttgattgtgctttgtaactgccttcgcctccttccctgctgctgtaagacactcgcctttctcgcggtcatgagcattggcgcgcacaccgttagtgcgtacgaacatgtaactgtgatccctaacacagttggcgttccatacaaaacgttggtgaaccgcccgggttacagtccaatggtgctcgagatggaacttctgagcgtcacactcgaacccaccttgagcctcgattacataacctgcgagtacaagacagttatccccagcccatatgtaaagtgttgtgggacggccgagtgcaaggataagaatcttcccgactactcatgcaaagtatttaccggcgtgtatcccttcatgtggggtggcgcatactgtttctgcgatgctgagaatacgcagttgtccgaggcgcatgtcgagaaatctgaaagctgtaagacggaatttgcgagtgcgtatagagcacatactgctagtgcgagcgcaaagcttcgagtactctatcaaggcaacaatataacggtcaccgcttatgcaaatggcgaccatgcagttactgtgaaggatgcgaaatttattgtcggacccatgagtagcgcgtggacaccgtttgataacaagatagttgtctacaaaggggacgtatataatatggactacccgccctttggtgcagggcgacctgggcagtttggggatattcagtcacggacgcccgagtcaaaggatgtctatgcaaatacacaattggtgcttcaacgacccgctgcgggtaccgtacacgtaccttactctcaagccccctcaggcttcaaatactggttgaaggagagaggcgcgagtttgcaacataccgcgccgtttggatgtcaaattgcgaccaacccggtgcgggcgatgaattgtgcagttggaaacatgccgatctcaatagatatacctgacgcagctttcacccgggttgtcgatgcgccttcactgaccgatatgtcttgtgaggtgcctgcctgtacccatagctctgattttgggggcgtggcgattatcaagtatgcagtctccaaaaagggtaaatgcgcagtgcacagcatgacaaatgctgttaccatacgagaggcagagatcgaagtagaggggaactcacaacttcagatctcatttagtacagccctcgcgagtgcggagttccgagtgcaggtgtgttccacccaggtccactgcgctgccgaatgccatcccccgaaagaccatatagtgaattaccctgccagccataccacattgggcgtgcaagatatatcagctactgcaatgagttgggttcagaaaataacggggggggtaggccttgtcgtggccgtagcggctctgatcttgatcgtagtcctgtgcgtatctttttctcggcactaa

SEQ ID NO: 10 - protein, 981 aa, Chikungunya virus
  MISC_FEATURE (1)..(981): Amino acid sequence
MCLLANTTFPCSQPPCIPCCYEKEPEETLRMLEDNVMRPGYYQLLQASLTCSPHRQRRSTKDNFNVYKATRPYLAHCPDCGEGHSCHSPVALERIRNEATDGTLKIQVSLQIGIGTDDSHDWTKLRYMDNHIPADAGRAGLFVRTSAPCTITGTMGHFILARCPKGETLTVGFTDSRKISHSCTHPFHHDPPVIGREKFHSRPQHGKELPCSTYVQSNAATAEEIEVHMPPDTPDRTLLSQQSGNVKITVNSQTVRYKCNCGGSNEGLITTDKVINNCKVDQCHAAVTNHKKWQYNSPLVPRNAELGDRKGKIHIPFPLANVTCMVPKARNPTVTYGKNQVIMLLYPDHPTLLSYRSMGEEPNYQEEWVTHKKEVVLTVPTEGLEVTWGNNEPYKYWPQLSANGTAHGHPHEIILYYYELYPTMTVVVVSVASFILLSMVGMAVGMCMCARRRCITPYELTPGATVPFLLSLICCIRTAKAATYQEAAVYLWNEQQPLFWLQALIPLAALIVLCNCLRLLPCCCKTLAFLAVMSIGAHTVSAYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAMNCAVGNMPISIDIPDAAFTRVVDAPSLTDMSCEVPACTHSSDFGGVAIIKYAVSKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 11 - protein, 15 aa, Artificial Sequence
  Synthetic Linker
GGGGSGGGGSGGGGS

SEQ ID NO: 12 - protein, 16 aa, Artificial Sequence
  Synthetic Linker
GGGGSGGGGSGGGGSS

SEQ ID NO: 13 - protein, 11 aa, Artificial Sequence
  Synthetic Linker
GGGGSGGGGSS

SEQ ID NO: 14 - protein, 5 aa, Artificial Sequence
  Synthetic Linker
GGGGS

SEQ ID NO: 15 - DNA, 15 bp, Homo sapiens
  MISC_FEATURE (1)..(15): Human
ggtggtggcggcagt

SEQ ID NO: 16 - DNA, 15 bp, Mus musculus
  MISC_FEATURE (1)..(15): Mouse
ggagggggaggcagc

SEQ ID NO: 17 - DNA, 45 bp, Homo sapiens
  MISC_FEATURE (1)..(45): Human encoding SEQ ID NO11
ggaggcggcggtagcggaggtggcggctccggaggtggggggtcc

SEQ ID NO: 18 - DNA, 45 bp, Mus musculus
  MISC_FEATURE (1)..(45): Mouse encoding SEQ ID NO11
gggggaggtggctccggcggcggcgggtccggaggtggaggtagc

SEQ ID NO: 19 - protein, 19 aa, Artificial Sequence
  Synthetic Linker
GGGGSGGGGSGGGLASGGS

SEQ ID NO: 20 - DNA, 57 bp, Homo sapiens
  MISC_FEATURE (1)..(57): Human encoding SEQ ID NO19
ggcggtggtggatcaggaggaggcggaagcggtggaggcagtgcctcaggaggctct

SEQ ID NO: 21 - DNA, 57 bp, Mus musculus
  MISC_FEATURE (1)..(57): Mouse encoding SEQ ID NO19
ggaggtggaggatcaggaggagggggctctggcggcggtagcgcaagtggcggctcc

SEQ ID NO: 22 - DNA, 45 bp, Artificial Sequence
  Synthetic Linker
ggtggcggtggctcgggcggtggtgggtcgggtggcggcggatct

SEQ ID NO: 23 - DNA, 1791 bp, Homo sapiens
  MISC_FEATURE (1)..(1791): Human Single-Chain Secreted IL12 Wild-Type
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggctcgggcggtggtgggtcgggtggcggcggatctatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 24 - protein, 596 aa, Homo sapiens
  MISC_FEATURE (1)..(596): Human Single-Chain Secreted IL12 Wild-Type Amino acid
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSMWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 25 - DNA, 144 bp, Homo sapiens
  MISC_FEATURE (1)..(144): B7.1 (human, codon-optimized)
gacaatttgttgcccagttgggcgattacccttatatcagttaatggcatatttgttatttgctgtctcacttactgcttcgccccaaggtgccgcgagagacgccgaaatgaaaggctgaggcgagagtcagtgcggccagtc

SEQ ID NO: 26 - protein, 48 aa, Homo sapiens
  MISC_FEATURE (1)..(48): B7.1 (human, codon-optimized)
DNLLPSWAITLISVNGIFVICCLTYCFAPRCRERRRNERLRRESVRPV

SEQ ID NO: 27 - DNA, 210 bp, Mus musculus
  MISC_FEATURE (1)..(210): B7.1 (mouse, codon-optimized)
ccccctgaagaccctccagacagtaagaatacacttgtgttgtttggcgctggattcggagcagtaataaccgttgtagttattgtggttatcattaaatgtttttgtaagcatcgctcctgttttagaaggaacgaggcatcccgagaaactaataatagtttgacattcggacccgaggaggcattggcagaacagacagtctttctc

SEQ ID NO: 28 - protein, 70 aa, Mus musculus
  MISC_FEATURE (1)..(210): B7.1 (mouse, codon-optimized)
PPEDPPDSKNTLVLFGAGFGAVITVVVIVVIIKCFCKHRSCFRRNEASRETNNSLTFGPEEALAEQTVFL

SEQ ID NO: 29 - DNA, 69 bp, Homo sapiens
  MISC_FEATURE (1)..(69): CD28 (human, codon-optimized)
gtcctcgttgtcgtgggtggtgtactggcttgttattcacttcttgtaaccgttgcgttcataattttc

SEQ ID NO: 30 - protein, 23 aa, Homo sapiens
  MISC_FEATURE (1)..(23): CD28 (human, codon-optimized)
VLVVVGGVLACYSLLVTVAFIIF

SEQ ID NO: 31 - DNA, 81 bp, Mus musculus
  MISC_FEATURE (1)..(81): CD28 (mouse, codon-optimized)
ttttgggcactcgtagtagttgctggtgtgttgttttgctacggactgctcgtaactgtcgcactttgcgtcatttggact

SEQ ID NO: 32 - protein, 27 aa, Mus musculus
  MISC_FEATURE (1)..(27): CD28 (mouse, codon-optimized)
FWALVVVAGVLFCYGLLVTVALCVIWT

SEQ ID NO: 33 - DNA, 63 bp, Homo sapiens
  MISC_FEATURE (1)..(63): CD8 (human, codon-optimized)
atatacatctgggccccactcgccggaacgtgcggcgtcttgcttctcagcctggtgatcaca

SEQ ID NO: 34 - protein, 21 aa, Homo sapiens
  MISC_FEATURE (1)..(21): CD8 (human, codon-optimized)
IYIWAPLAGTCGVLLLSLVIT

SEQ ID NO: 35 - DNA, 63 bp, Mus musculus
  MISC_FEATURE (1)..(63): CD8 (mouse, codon-optimized)
atatgggcacccctggctggcatttgtgttgcattgcttttgtcactgataattaccttgatt

SEQ ID NO: 36 - protein, 21 aa, Mus musculus
  MISC_FEATURE (1)..(21): CD8 (mouse, codon-optimized)
IWAPLAGICVALLLSLIITLI

SEQ ID NO: 37 - DNA, 1935 bp, Homo sapiens
  MISC_FEATURE (1)..(1935): Human Membrane-Anchored IL12
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggctcgggcggtggtgggtcgggtggcggcggatctatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccgacaatttgttgcccagttgggcgattacccttatatcagttaatggcatatttgttatttgctgtctcacttactgcttcgccccaaggtgccgcgagagacgccgaaatgaaaggctgaggcgagagtcagtgcggccagtctaa

SEQ ID NO: 38 - protein, 644 aa, Homo sapiens
  MISC_FEATURE (1)..(644): Human Membrane-Anchored IL12
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSMWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASDNLLPSWAITLISVNGIFVICCLTYCFAPRCRERRRNERLRRESVRPV

SEQ ID NO: 39 - DNA, 1773 bp, Mus musculus
  MISC_FEATURE (1)..(1773): Mouse Secreted Single-Chain IL-12
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcggcggcgggagtggcggcgggggttctggcggaggcctcgctagcggtggctccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 40 - protein, 590 aa, Mus musculus
  MISC_FEATURE (1)..(590): Mouse Secreted Single-Chain IL-12
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGLASGGSMVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 41 - DNA, 1983 bp, Mus musculus
  MISC_FEATURE (1)..(1983): Mouse Membrane-Anchored Single-Chain IL-12
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcggcggcgggagtggcggcgggggttctggcggaggcctcgctagcggtggctccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccccccctgaagaccctccagacagtaagaatacacttgtgttgtttggcgctggattcggagcagtaataaccgttgtagttattgtggttatcattaaatgtttttgtaagcatcgctcctgttttagaaggaacgaggcatcccgagaaactaataatagtttgacattcggacccgaggaggcattggcagaacagacagtctttctctga

SEQ ID NO: 42 - protein, 660 aa, Mus musculus
  MISC_FEATURE (1)..(660): Mouse Membrane-Anchored Single-Chain IL-12
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGLASGGSMVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAPPEDPPDSKNTLVLFGAGFGAVITVVVIVVIIKCFCKHRSCFRRNEASRETNNSLTFGPEEALAEQTVFL

SEQ ID NO: 43 - DNA, 786 bp, Homo sapiens
  MISC_FEATURE (1)..(786): Human Secreted CD40L
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 44 - protein, 261 aa, Homo sapiens
  MISC_FEATURE (1)..(261): Human Secreted CD40L
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 45 - DNA, 765 bp, Homo sapiens
  MISC_FEATURE (1)..(765): Human Membrane-Anchored CD40L
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 46 - protein, 254 aa, Homo sapiens
  MISC_FEATURE (1)..(254): Human Membrane-Anchored CD40L
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 47 - DNA, 21 bp, Artificial Sequence
  Synthetic Deleted
agctttgaaatgcaaaaaggt

SEQ ID NO: 48 - DNA, 783 bp, Mus musculus
  MISC_FEATURE (1)..(783): Mouse Secreted CD40L
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctga

SEQ ID NO: 49 - protein, 260 aa, Mus musculus
  MISC_FEATURE (1)..(260): Mouse Secreted CD40L
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 50 - DNA, 762 bp, Mus musculus
  MISC_FEATURE (1)..(762): Mouse Membrane-anchored CD40L
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctga

SEQ ID NO: 51 - protein, 253 aa, Mus musculus
  MISC_FEATURE (1)..(253): Mouse Membrane-anchored CD40L
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 52 - DNA, 20 bp, Artificial Sequence
  Synthetic Deleted
gctttgaaatgcaaagaggt

SEQ ID NO: 53 - DNA, 13011 bp, Artificial Sequence; identical to SEQ ID NO: 1
  Synthetic VSV-DELTA-G-LASV wild-type secreted IL-12 (between positions 4 and 5

SEQ ID NO: 54 - DNA, 12006 bp, Homo sapiens
  MISC_FEATURE (1)..(12006): VSV-DELTA-G-LASV Human CD40L (between positions 4 and 5)
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaactaacagagatcgatctgtttccttgacactatgggtcagatcgttactttcttccaggaagtaccccatgttatagaagaggttatgaatatagtccttattgctttgagcgtcttggcggtgctgaaaggtctctacaactttgcaacttgtggtctggtcggcctggtgactttcctcctgctgtgtggccggtcctgcacgactagcttgtacaaaggggtctatgagttgcagactctcgagcttaatatggaaacattgaacatgacaatgccgctctcatgcacaaagaacaactcccaccactatataatggtgggaaatgagactgggttggaacttacgctgaccaacactagtattattaaccataagttctgtaacctctctgatgcccacaagaaaaatctctatgatcatgcgttgatgtcaataataagcacatttcatctgtctatacctaattttaaccaatatgaagctatgtcttgtgatttcaacgggggtaagatatccgtccaatataatttgtctcactcctacgctggagatgctgcaaaccattgcggcactgttgccaacggagttctgcaaaccttcatgcggatggcatggggtggtagttacatcgccttggattctgggagaggcaattgggactgcattatgacttcctaccagtacctcattattcaaaatacgacgtgggaggatcattgccaattttctaggcccagcccgattgggtacctcggactcctttcccaacggactagggatatatacattagtcggaggctgcttgggaccttcacatggacactctctgactcagaaggcaaggatacgcccggtggatactgtttgaccagatggatgcttattgaggccgagcttaaatgctttggtaataccgccgtagcgaagtgtaatgaaaaacacgacgaggaattctgcgacatgctcaggttgttcgattttaacaaacaggctatacaacgcctgaaagcagaggcccaaatgtctatccaactcatcaacaaggcagtcaatgcactcattaatgatcagctgattatgaagaaccacctgagggatataatgggcattccttactgcaactattctaagtattggtatctgaaccatacaactacgggacggacgtccctgcctaaatgttggcttgtcagtaatggatcctacctcaatgagacacacttctctgacgacatagaacaacaggcggataatatgattactgagatgttgcagaaagagtatatggagagacagggcaaaacaccgcttggcctcgtggacctgtttgtcttctctacttccttctatctcatttctatctttcttcatcttgttaagatcccgacccacaggcatatagtgggcaagagttgtccaaagccccaccggctgaatcatatgggcatttgcagttgtggtctgtacaaacagcccggagtgcccgtcaagtggaaaaggtgactcaaatcctgcacaacagattcttcatgtttggaccaaatcaacttgtgataccatgctcaaagaggcctcaattatatttgagtttttaatttttatgaaaaaaactaacagcaatcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgactcaaatcctgcacaacagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 55 - DNA, 13155 bp, Artificial Sequence
  Synthetic VSV-LASV-IL12 (Codon-Optimized)
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaactaacagagatcgatctgtttccttgacactatgggtcagatcgttactttcttccaggaagtaccccatgttatagaagaggttatgaatatagtccttattgctttgagcgtcttggcggtgctgaaaggtctctacaactttgcaacttgtggtctggtcggcctggtgactttcctcctgctgtgtggccggtcctgcacgactagcttgtacaaaggggtctatgagttgcagactctcgagcttaatatggaaacattgaacatgacaatgccgctctcatgcacaaagaacaactcccaccactatataatggtgggaaatgagactgggttggaacttacgctgaccaacactagtattattaaccataagttctgtaacctctctgatgcccacaagaaaaatctctatgatcatgcgttgatgtcaataataagcacatttcatctgtctatacctaattttaaccaatatgaagctatgtcttgtgatttcaacgggggtaagatatccgtccaatataatttgtctcactcctacgctggagatgctgcaaaccattgcggcactgttgccaacggagttctgcaaaccttcatgcggatggcatggggtggtagttacatcgccttggattctgggagaggcaattgggactgcattatgacttcctaccagtacctcattattcaaaatacgacgtgggaggatcattgccaattttctaggcccagcccgattgggtacctcggactcctttcccaacggactagggatatatacattagtcggaggctgcttgggaccttcacatggacactctctgactcagaaggcaaggatacgcccggtggatactgtttgaccagatggatgcttattgaggccgagcttaaatgctttggtaataccgccgtagcgaagtgtaatgaaaaacacgacgaggaattctgcgacatgctcaggttgttcgattttaacaaacaggctatacaacgcctgaaagcagaggcccaaatgtctatccaactcatcaacaaggcagtcaatgcactcattaatgatcagctgattatgaagaaccacctgagggatataatgggcattccttactgcaactattctaagtattggtatctgaaccatacaactacgggacggacgtccctgcctaaatgttggcttgtcagtaatggatcctacctcaatgagacacacttctctgacgacatagaacaacaggcggataatatgattactgagatgttgcagaaagagtatatggagagacagggcaaaacaccgcttggcctcgtggacctgtttgtcttctctacttccttctatctcatttctatctttcttcatcttgttaagatcccgacccacaggcatatagtgggcaagagttgtccaaagccccaccggctgaatcatatgggcatttgcagttgtggtctgtacaaacagcccggagtgcccgtcaagtggaaaaggtgactcaaatcctgcacaacagattcttcatgtttggaccaaatcaacttgtgataccatgctcaaagaggcctcaattatatttgagtttttaatttttatgaaaaaaactaacagcaatcatgtgtcaccagcaactcgtgattagttggtttagtttggtattcctcgccagccccctcgtcgccatatgggaattgaagaaagatgtctatgtcgtcgaacttgattggtaccccgacgcgcccggagagatggttgttcttacgtgtgatactcccgaggaagacggtataacatggacgctggatcaatcatccgaggtactgggatcaggcaaaactctcaccatacaagtgaaggagttcggcgacgctggtcagtacacgtgtcacaaaggtggtgaggttttgagtcattctctgttgcttttgcataaaaaagaagatggtatttggtcaaccgacattctgaaggaccaaaaagagcccaaaaacaaaaccttcctgcgatgtgaagccaaaaactactctggacggttcacatgttggtggctgacgaccataagtacggacctgactttttctgttaaatctagcagagggtcatccgacccccaaggcgtgacgtgtggcgcagcgacgctcagcgccgagcgggttcgcggagacaacaaggaatacgagtactctgttgagtgccaggaagattctgcctgtcctgcagccgaagagagtcttcccatagaggtcatggttgatgcagtccacaagctgaaatatgagaattatacaagcagcttctttatccgagatatcatcaagccggatccacccaagaatctccaacttaaacccctgaagaatagtcgccaggtcgaagtcagctgggagtatcccgatacatggtcaacaccgcattcttattttagtttgacattttgcgtccaagtacagggcaaaagtaaacgcgagaaaaaggaccgagtattcacagacaaaactagcgcaacggtcatatgtcggaagaacgcgagcataagtgttagagctcaagataggtactatagttcatcctggtctgagtgggcctctgtcccatgtagcggtggggggggatctggggggggtgggagtggaggtggaggttcaatgtggccaccagggtcagctagtcaaccacctccatcacccgctgctgcaaccggtttgcatccggctgcccgcccagtgtcccttcaatgtagactgagtatgtgcccagcgcgctccctcttgcttgttgccacccttgttctgctcgatcatttgtccctcgcacgcaatctgcccgttgccaccccggatcccggaatgttcccctgtttgcaccattcccaaaatcttcttcgggcggtctccaatatgctgcaaaaagcaaggcaaacccttgaattttacccatgcacgagcgaagaaattgatcatgaggacatcaccaaggataaaacttcaacagtggaagcttgtttgccattggagctgactaagaatgaaagttgtctcaatagtcgagaaacgagtttcataacgaatggatcatgcctcgcatcaagaaagacttctttcatgatggctctgtgtctctcctccatctacgaggaccttaagatgtatcaagtcgagtttaaaaccatgaacgcgaaattgctgatggatcctaagcggcagatttttctcgaccaaaatatgttggcagttatcgatgagctcatgcaagcccttaactttaacagcgagacagtcccccaaaagtcctctcttgaagaaccagacttctacaaaacaaaaattaagctctgcatactgctccacgcttttcggattagagcagtaacaatagaccgagtaatgtcatacctgaatgccagtgataatctcttgccatcttgggccattactctgatttccgttaatggtattttcgtcatatgctgtctgacatactgttttgcgccgcgctgccgggaaagacgcaggaatgagcgactgcggcgggagtcagttcgcccagtgtaactcaaatcctgcacaacagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 56 - DNA, 11985 bp, Artificial Sequence
  Synthetic VSV-Lassa-CD40L Codon Optimized
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaactaacagagatcgatctgtttccttgacactatgggtcagatcgttactttcttccaggaagtaccccatgttatagaagaggttatgaatatagtccttattgctttgagcgtcttggcggtgctgaaaggtctctacaactttgcaacttgtggtctggtcggcctggtgactttcctcctgctgtgtggccggtcctgcacgactagcttgtacaaaggggtctatgagttgcagactctcgagcttaatatggaaacattgaacatgacaatgccgctctcatgcacaaagaacaactcccaccactatataatggtgggaaatgagactgggttggaacttacgctgaccaacactagtattattaaccataagttctgtaacctctctgatgcccacaagaaaaatctctatgatcatgcgttgatgtcaataataagcacatttcatctgtctatacctaattttaaccaatatgaagctatgtcttgtgatttcaacgggggtaagatatccgtccaatataatttgtctcactcctacgctggagatgctgcaaaccattgcggcactgttgccaacggagttctgcaaaccttcatgcggatggcatggggtggtagttacatcgccttggattctgggagaggcaattgggactgcattatgacttcctaccagtacctcattattcaaaatacgacgtgggaggatcattgccaattttctaggcccagcccgattgggtacctcggactcctttcccaacggactagggatatatacattagtcggaggctgcttgggaccttcacatggacactctctgactcagaaggcaaggatacgcccggtggatactgtttgaccagatggatgcttattgaggccgagcttaaatgctttggtaataccgccgtagcgaagtgtaatgaaaaacacgacgaggaattctgcgacatgctcaggttgttcgattttaacaaacaggctatacaacgcctgaaagcagaggcccaaatgtctatccaactcatcaacaaggcagtcaatgcactcattaatgatcagctgattatgaagaaccacctgagggatataatgggcattccttactgcaactattctaagtattggtatctgaaccatacaactacgggacggacgtccctgcctaaatgttggcttgtcagtaatggatcctacctcaatgagacacacttctctgacgacatagaacaacaggcggataatatgattactgagatgttgcagaaagagtatatggagagacagggcaaaacaccgcttggcctcgtggacctgtttgtcttctctacttccttctatctcatttctatctttcttcatcttgttaagatcccgacccacaggcatatagtgggcaagagttgtccaaagccccaccggctgaatcatatgggcatttgcagttgtggtctgtacaaacagcccggagtgcccgtcaagtggaaaaggtgactcaaatcctgcacaacagattcttcatgtttggaccaaatcaacttgtgataccatgctcaaagaggcctcaattatatttgagtttttaatttttatgaaaaaaactaacagcaatcatgatagaaacctataatcagacgagtcctagatccgctgctacaggtcttcctatttcaatgaagatattcatgtatttgctgacggtctttctgatcacgcaaatgatcggaagcgcgctgtttgccgtgtatctccaccgaaggttggacaaaattgaagatgagcgaaatctccatgaagacttcgtttttatgaagactattcagcgatgtaacacaggggagagatcactctcccttcttaattgcgaagaaattaagagccaattcgaaggcttcgtgaaagatattatgttgaacaaagaagaaacgaaaaaagagaacgatcagaacccccagatcgcggcccatgttatctctgaagcttcttccaagacaacctccgtactccaatgggcagagaagggatactataccatgtccaacaaccttgtaacacttgagaacgggaagcaactgacagtgaagagacaagggctctactatatttacgctcaagttaccttttgtagcaaccgcgaggcctcaagccaagctccgttcatagccagcctctgtctcaagagtcccggacggtttgaacgcattcttctgagggccgcaaatacacattctagtgccaaaccttgcggccagcagtcaatacatttgggcggcgttttcgagctgcaacctggggccagtgtcttcgtaaacgtcacagacccatctcaagtttcccatggaacgggattcacgtccttcggtctgcttaaactttgactcaaatcctgcacaacagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 57 - DNA, 14058 bp, Artificial Sequence
  Synthetic VSV-LASSA-IL12-CD40L Codon Optimized
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaactaacagagatcgatctgtttccttgacactatgtgtcaccagcaactcgtgattagttggtttagtttggtattcctcgccagccccctcgtcgccatatgggaattgaagaaagatgtctatgtcgtcgaacttgattggtaccccgacgcgcccggagagatggttgttcttacgtgtgatactcccgaggaagacggtataacatggacgctggatcaatcatccgaggtactgggatcaggcaaaactctcaccatacaagtgaaggagttcggcgacgctggtcagtacacgtgtcacaaaggtggtgaggttttgagtcattctctgttgcttttgcataaaaaagaagatggtatttggtcaaccgacattctgaaggaccaaaaagagcccaaaaacaaaaccttcctgcgatgtgaagccaaaaactactctggacggttcacatgttggtggctgacgaccataagtacggacctgactttttctgttaaatctagcagagggtcatccgacccccaaggcgtgacgtgtggcgcagcgacgctcagcgccgagcgggttcgcggagacaacaaggaatacgagtactctgttgagtgccaggaagattctgcctgtcctgcagccgaagagagtcttcccatagaggtcatggttgatgcagtccacaagctgaaatatgagaattatacaagcagcttctttatccgagatatcatcaagccggatccacccaagaatctccaacttaaacccctgaagaatagtcgccaggtcgaagtcagctgggagtatcccgatacatggtcaacaccgcattcttattttagtttgacattttgcgtccaagtacagggcaaaagtaaacgcgagaaaaaggaccgagtattcacagacaaaactagcgcaacggtcatatgtcggaagaacgcgagcataagtgttagagctcaagataggtactatagttcatcctggtctgagtgggcctctgtcccatgtagcggtggggggggatctggggggggtgggagtggaggtggaggttcaatgtggccaccagggtcagctagtcaaccacctccatcacccgctgctgcaaccggtttgcatccggctgcccgcccagtgtcccttcaatgtagactgagtatgtgcccagcgcgctccctcttgcttgttgccacccttgttctgctcgatcatttgtccctcgcacgcaatctgcccgttgccaccccggatcccggaatgttcccctgtttgcaccattcccaaaatcttcttcgggcggtctccaatatgctgcaaaaagcaaggcaaacccttgaattttacccatgcacgagcgaagaaattgatcatgaggacatcaccaaggataaaacttcaacagtggaagcttgtttgccattggagctgactaagaatgaaagttgtctcaatagtcgagaaacgagtttcataacgaatggatcatgcctcgcatcaagaaagacttctttcatgatggctctgtgtctctcctccatctacgaggaccttaagatgtatcaagtcgagtttaaaaccatgaacgcgaaattgctgatggatcctaagcggcagatttttctcgaccaaaatatgttggcagttatcgatgagctcatgcaagcccttaactttaacagcgagacagtcccccaaaagtcctctcttgaagaaccagacttctacaaaacaaaaattaagctctgcatactgctccacgcttttcggattagagcagtaacaatagaccgagtaatgtcatacctgaatgccagtgataatctcttgccatcttgggccattactctgatttccgttaatggtattttcgtcatatgctgtctgacatactgttttgcgccgcgctgccgggaaagacgcaggaatgagcgactgcggcgggagtcagttcgcccagtgtaagctagtctaacttctagcttctgaacaatccccggtttactcagtctctcctaattccagcctctcgaacaactaatatcctgtcttttctatccctatgaaaaaaactaacagagatcgatctgtttccttgacactatgggtcagatcgttactttcttccaggaagtaccccatgttatagaagaggttatgaatatagtccttattgctttgagcgtcttggcggtgctgaaaggtctctacaactttgcaacttgtggtctggtcggcctggtgactttcctcctgctgtgtggccggtcctgcacgactagcttgtacaaaggggtctatgagttgcagactctcgagcttaatatggaaacattgaacatgacaatgccgctctcatgcacaaagaacaactcccaccactatataatggtgggaaatgagactgggttggaacttacgctgaccaacactagtattattaaccataagttctgtaacctctctgatgcccacaagaaaaatctctatgatcatgcgttgatgtcaataataagcacatttcatctgtctatacctaattttaaccaatatgaagctatgtcttgtgatttcaacgggggtaagatatccgtccaatataatttgtctcactcctacgctggagatgctgcaaaccattgcggcactgttgccaacggagttctgcaaaccttcatgcggatggcatggggtggtagttacatcgccttggattctgggagaggcaattgggactgcattatgacttcctaccagtacctcattattcaaaatacgacgtgggaggatcattgccaattttctaggcccagcccgattgggtacctcggactcctttcccaacggactagggatatatacattagtcggaggctgcttgggaccttcacatggacactctctgactcagaaggcaaggatacgcccggtggatactgtttgaccagatggatgcttattgaggccgagcttaaatgctttggtaataccgccgtagcgaagtgtaatgaaaaacacgacgaggaattctgcgacatgctcaggttgttcgattttaacaaacaggctatacaacgcctgaaagcagaggcccaaatgtctatccaactcatcaacaaggcagtcaatgcactcattaatgatcagctgattatgaagaaccacctgagggatataatgggcattccttactgcaactattctaagtattggtatctgaaccatacaactacgggacggacgtccctgcctaaatgttggcttgtcagtaatggatcctacctcaatgagacacacttctctgacgacatagaacaacaggcggataatatgattactgagatgttgcagaaagagtatatggagagacagggcaaaacaccgcttggcctcgtggacctgtttgtcttctctacttccttctatctcatttctatctttcttcatcttgttaagatcccgacccacaggcatatagtgggcaagagttgtccaaagccccaccggctgaatcatatgggcatttgcagttgtggtctgtacaaacagcccggagtgcccgtcaagtggaaaaggtgactcaaatcctgcacaacagattcttcatgtttggaccaaatcaacttgtgataccatgctcaaagaggcctcaattatatttgagtttttaatttttatgaaaaaaactaacagcaatcatgatagaaacctataatcagacgagtcctagatccgctgctacaggtcttcctatttcaatgaagatattcatgtatttgctgacggtctttctgatcacgcaaatgatcggaagcgcgctgtttgccgtgtatctccaccgaaggttggacaaaattgaagatgagcgaaatctccatgaagacttcgtttttatgaagactattcagcgatgtaacacaggggagagatcactctcccttcttaattgcgaagaaattaagagccaattcgaaggcttcgtgaaagatattatgttgaacaaagaagaaacgaaaaaagagaacgatcagaacccccagatcgcggcccatgttatctctgaagcttcttccaagacaacctccgtactccaatgggcagagaagggatactataccatgtccaacaaccttgtaacacttgagaacgggaagcaactgacagtgaagagacaagggctctactatatttacgctcaagttaccttttgtagcaaccgcgaggcctcaagccaagctccgttcatagccagcctctgtctcaagagtcccggacggtttgaacgcattcttctgagggccgcaaatacacattctagtgccaaaccttgcggccagcagtcaatacatttgggcggcgttttcgagctgcaacctggggccagtgtcttcgtaaacgtcacagacccatctcaagtttcccatggaacgggattcacgtccttcggtctgcttaaactttgactcaaatcctgcacaacagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 58 - DNA, 1791 bp, Homo sapiens
  MISC_FEATURE (1)..(1791): Human Codon-Optimized
atgtgtcatcaacaattggtgatatcctggttctccttggtttttttggcctcaccgcttgttgctatttgggagctcaaaaaagatgtgtatgtggtggaactggactggtacccagatgcaccgggagagatggtagtactgacttgtgatacgccggaagaagatggcattacttggacgttggatcagagcagcgaggtattgggaagtggtaagaccctgactatccaggtcaaggagtttggtgatgctggtcagtacacgtgccacaaggggggtgaagtgctttcccactcattgcttctcctccacaaaaaagaagatggcatttggagtacagacatattgaaggatcagaaagaaccgaagaacaagaccttcctccgatgtgaagcgaaaaattattctgggcgcttcacctgctggtggttgaccactatctcaacggacttgactttcagtgttaaaagtagccgcgggtcttccgatccccaaggagtcacttgtggagccgctaccctgtctgcagaacgcgtacgaggtgacaacaaagagtacgaatatagcgttgaatgccaggaagatagcgcgtgtccagcggccgaagagtcactgcctattgaggtcatggtggacgccgttcataagcttaagtacgaaaattacacaagctcttttttcattcgggacataatcaaacccgatccccccaaaaaccttcaactcaagcccttgaagaactctcgacaggtcgaagtaagttgggaatacccggacacatggagcactccccacagttacttcagtttgaccttctgtgtgcaggtgcagggaaaaagcaaacgagaaaagaaagaccgggtgtttacggataagacaagtgcgactgtaatctgtcgcaagaacgcaagcatcagcgtacgcgctcaggatcgatactattcaagctcttggtccgagtgggcctccgtaccgtgttcaggtggcggtggaagtgggggtggcgggtccggaggtggtggaagtatgtggcctccgggctctgccagtcagccaccgccatccccggcggcagctactggccttcacccagcagcacggcctgtatcccttcagtgcaggctttctatgtgcccagcccgctccttgctcttggtagcgaccttggtgttgttggaccacttgtccctcgcgagaaatctcccagttgccacccccgacccagggatgttcccttgtctccatcattcccagaaccttcttcgggcagtttctaacatgctgcaaaaggcccggcagaccttggagttttatccctgtacttccgaggaaatagaccatgaagatataaccaaggataagacgagcacggtagaggcgtgcctgcccctggaacttaccaaaaatgaaagctgtcttaactcccgggagacgagtttcattaccaacggttcctgcttggcctcccggaagacgagcttcatgatggctctgtgcctgtccagtatctatgaagacctcaaaatgtaccaagtggaattcaaaaccatgaacgcgaagctgcttatggacccaaagcggcaaatctttctggatcaaaatatgctcgcggttatcgatgaactgatgcaggcactgaattttaatagcgaaaccgtcccgcaaaagtccagcctggaggaaccagacttctacaagactaagatcaagctgtgtatacttttgcacgcttttcggattagggcagtgacgatagaccgggttatgtcataccttaatgcgagctaa

SEQ ID NO: 59 - DNA, 1935 bp, Homo sapiens
  MISC_FEATURE (1)..(1935): Human Codon-Optimized Membrane-Anchored IL12
atgtgtcaccagcaactcgtgattagttggtttagtttggtattcctcgccagccccctcgtcgccatatgggaattgaagaaagatgtctatgtcgtcgaacttgattggtaccccgacgcgcccggagagatggttgttcttacgtgtgatactcccgaggaagacggtataacatggacgctggatcaatcatccgaggtactgggatcaggcaaaactctcaccatacaagtgaaggagttcggcgacgctggtcagtacacgtgtcacaaaggtggtgaggttttgagtcattctctgttgcttttgcataaaaaagaagatggtatttggtcaaccgacattctgaaggaccaaaaagagcccaaaaacaaaaccttcctgcgatgtgaagccaaaaactactctggacggttcacatgttggtggctgacgaccataagtacggacctgactttttctgttaaatctagcagagggtcatccgacccccaaggcgtgacgtgtggcgcagcgacgctcagcgccgagcgggttcgcggagacaacaaggaatacgagtactctgttgagtgccaggaagattctgcctgtcctgcagccgaagagagtcttcccatagaggtcatggttgatgcagtccacaagctgaaatatgagaattatacaagcagcttctttatccgagatatcatcaagccggatccacccaagaatctccaacttaaacccctgaagaatagtcgccaggtcgaagtcagctgggagtatcccgatacatggtcaacaccgcattcttattttagtttgacattttgcgtccaagtacagggcaaaagtaaacgcgagaaaaaggaccgagtattcacagacaaaactagcgcaacggtcatatgtcggaagaacgcgagcataagtgttagagctcaagataggtactatagttcatcctggtctgagtgggcctctgtcccatgtagcggtggggggggatctggggggggtgggagtggaggtggaggttcaatgtggccaccagggtcagctagtcaaccacctccatcacccgctgctgcaaccggtttgcatccggctgcccgcccagtgtcccttcaatgtagactgagtatgtgcccagcgcgctccctcttgcttgttgccacccttgttctgctcgatcatttgtccctcgcacgcaatctgcccgttgccaccccggatcccggaatgttcccctgtttgcaccattcccaaaatcttcttcgggcggtctccaatatgctgcaaaaagcaaggcaaacccttgaattttacccatgcacgagcgaagaaattgatcatgaggacatcaccaaggataaaacttcaacagtggaagcttgtttgccattggagctgactaagaatgaaagttgtctcaatagtcgagaaacgagtttcataacgaatggatcatgcctcgcatcaagaaagacttctttcatgatggctctgtgtctctcctccatctacgaggaccttaagatgtatcaagtcgagtttaaaaccatgaacgcgaaattgctgatggatcctaagcggcagatttttctcgaccaaaatatgttggcagttatcgatgagctcatgcaagcccttaactttaacagcgagacagtcccccaaaagtcctctcttgaagaaccagacttctacaaaacaaaaattaagctctgcatactgctccacgcttttcggattagagcagtaacaatagaccgagtaatgtcatacctgaatgccagtgataatctcttgccatcttgggccattactctgatttccgttaatggtattttcgtcatatgctgtctgacatactgttttgcgccgcgctgccgggaaagacgcaggaatgagcgactgcggcgggagtcagttcgcccagtgtaa

SEQ ID NO: 60 - DNA, 1983 bp, Mus musculus
  MISC_FEATURE (1)..(1983): Mouse Codon Optimized Membrane-Anchored Single- Chain IL-12
atgtgcccacagaagctcaccataagctggttcgccatcgtgctcctggtaagccccctcatggcaatgtgggaactggagaaagatgtgtatgtcgttgaagtggattggacacctgatgctcctggcgagactgtcaatcttacctgcgacacccccgaagaggacgacataacatggactagtgatcagcgccatggggtgatcgggtccggaaagacccttacaataactgtcaaggagtttctcgatgccgggcaatacacttgtcataagggtggtgaaactctgagccatagtcatttgttgttgcataaaaaggaaaatgggatttggtccactgaaattctcaagaatttcaagaacaagacatttctgaaatgcgaggcccccaattatagtggacgctttacttgttcttggctggtccagagaaacatggaccttaagttcaatatcaaatcttcttcttcctctccagactcaagagcagtgacctgtgggatggcctctctctctgctgaaaaggtgaccctcgatcagcgggactacgaaaaatacagcgtgagctgccaagaggatgttacatgtcccactgccgaagagacacttccaatagagttggccctcgaggctagacaacagaacaaatatgagaattattccacatctttttttatccgggatatcatcaagcctgaccctcctaaaaatttgcaaatgaaacctcttaaaaatagtcaagttgaggtaagctgggagtaccctgatagctggtcaactccacatagctacttttcccttaagttctttgtgaggattcagagaaagaaggaaaagatgaaggagactgaggagggttgtaatcaaaagggtgcatttcttgttgaaaaaacttctacagaagtccagtgtaaaggtggcaacgtgtgcgtgcaggcacaagaccgctattacaactcatcttgttctaaatgggcttgtgttccatgcagggttaggtctggaggcggtggaagcggaggtggtggttcaggcggtggtctggctagtggtggtagtatggtgagcgtacccactgctagccccagcgcaagttcttcatcctcccagtgtcgctcaagtatgtgtcaatctcgctatctcttgtttctggctacacttgcactcctcaaccatctcagtctcgctcgggtgatccccgtaagtggtcccgcaagatgcctttctcaaagcaggaatctgctgaagacaaccgacgacatggtgaaaactgcaagggagaagttgaaacactattcctgtactgcagaagacatcgaccacgaagacataacccgagatcagacatcaactctcaagacttgccttccacttgaattgcataaaaacgaatcctgcctggcaaccagggagaccagcagtaccacacgaggctcttgtttgcctccccaaaaaacttctctcatgatgaccctttgtttgggatccatttacgaggatcttaagatgtaccaaactgagttccaagctataaatgctgcacttcaaaaccataaccatcaacaaataatcctcgacaaggggatgcttgtggcaatagatgaactcatgcagagccttaaccacaatggggagacattgcgccaaaaaccaccagtaggggaagccgacccctatagagtcaaaatgaaactctgcatattgctgcatgcatttagtaccagggtagtaaccatcaatcgagttatggggtacctctcaagcgcaccacctgaggatccaccagatagtaagaacacccttgtattgttcggggcaggctttggagccgtgatcacagtagtcgttatagttgttatcatcaaatgtttctgtaagcaccgaagttgtttccgacgaaacgaggcaagccgagaaaccaacaactctttgacttttggcccagaagaggcactggctgagcaaaccgtgtttttgtga

SEQ ID NO: 61 - DNA, 786 bp, Homo sapiens
  MISC_FEATURE (1)..(786): Human codon optimized secreted CD40L
atgattgaaacttataaccaaacaagcccgaggtctgcggctaccggactccccatttctatgaaaattttcatgtatttgctcactgtatttcttatcacgcagatgatcggaagcgccctcttcgccgtatacttgcacagaaggctggataaaattgaggatgaaagaaatctccatgaagactttgtcttcatgaagaccatccagaggtgcaatacaggcgaacgctctctgtctctgttgaattgcgaagagattaagagccagtttgagggcttcgtcaaggacataatgttgaacaaggaagagacgaaaaaagagaattcatttgaaatgcaaaaaggcgaccaaaacccccaaatcgcggcgcatgtgatctcagaagcctctagtaaaacaaccagcgttcttcaatgggctgagaagggatactacactatgtctaacaatcttgttactcttgagaacggcaagcaattgaccgtgaaaaggcagggcctgtattatatttacgcgcaggttacattctgttccaatagggaagcctcatcccaagctccgtttatcgcatctttgtgtctcaagtcccctggcaggttcgagcggatacttctcagagctgccaacacgcatagttcagctaaaccgtgcggacaacaaagcattcacctgggtggtgtgtttgagctgcaacctggcgcaagtgtcttcgtaaacgtcaccgatccctcccaagttagtcacgggacaggttttacatcatttggtctgttgaaattgtga

SEQ ID NO: 62 - DNA, 765 bp, Homo sapiens
  MISC_FEATURE (1)..(765): Human Codon optimized Membrane-Anchored CD40L
atgatagaaacctataatcagacgagtcctagatccgctgctacaggtcttcctatttcaatgaagatattcatgtatttgctgacggtctttctgatcacgcaaatgatcggaagcgcgctgtttgccgtgtatctccaccgaaggttggacaaaattgaagatgagcgaaatctccatgaagacttcgtttttatgaagactattcagcgatgtaacacaggggagagatcactctcccttcttaattgcgaagaaattaagagccaattcgaaggcttcgtgaaagatattatgttgaacaaagaagaaacgaaaaaagagaacgatcagaacccccagatcgcggcccatgttatctctgaagcttcttccaagacaacctccgtactccaatgggcagagaagggatactataccatgtccaacaaccttgtaacacttgagaacgggaagcaactgacagtgaagagacaagggctctactatatttacgctcaagttaccttttgtagcaaccgcgaggcctcaagccaagctccgttcatagccagcctctgtctcaagagtcccggacggtttgaacgcattcttctgagggccgcaaatacacattctagtgccaaaccttgcggccagcagtcaatacatttgggcggcgttttcgagctgcaacctggggccagtgtcttcgtaaacgtcacagacccatctcaagtttcccatggaacgggattcacgtccttcggtctgcttaaactttga

SEQ ID NO: 63 - DNA, 783 bp, Mus musculus
  MISC_FEATURE (1)..(783): Mouse Codon Optimized Secreted CD40L
atgattgaaacatacagtcagccatcccctaggtccgtggccactggccttcccgcctcaatgaaaatcttcatgtacttgctgacagtgttcctgataacacaaatgataggaagcgtgttgtttgctgtgtatctgcacagaagattggacaaagttgaagaggaggtgaatcttcacgaagacttcgtgttcatcaaaaaattgaaaaggtgcaacaaaggcgaaggcagcctctccttgttgaattgtgaagaaatgagacggcagttcgaggacctggtgaaagacatcaccttgaataaagaggaaaagaaagagaattcttttgagatgcagcgcggagacgaggacccccagattgctgcccacgtcgtgtcagaggctaatagcaatgcagcaagtgtactccagtgggcaaaaaaaggatattacaccatgaaaagtaatctcgtgatgttggagaatggtaaacagctgaccgttaagcgagagggactgtattatgtttatactcaagtaactttttgcagcaacagggagcctagtagtcagagacctttcattgttggtctctggctcaaaccatcatctgggtcagagaggatactgctgaaggccgcaaatactcatagttcaagtcaactttgcgagcaacagtccgttcacctggggggagttttcgagctgcaagctggagcctccgtgttcgttaatgtaactgaggctagtcaagtcattcaccgcgtaggtttctcatcttttggactgttgaagttgtga

SEQ ID NO: 64 - DNA, 762 bp, Mus musculus
  MISC_FEATURE (1)..(762): Mouse Codon-Optimized Membrane-Anchored CD40L
atgattgagacatattctcaaccaagtcccagatccgtcgcaacaggtctccccgcttcaatgaagatatttatgtatctgctgaccgtgttcctcattacccaaatgataggatcagtcctttttgctgtctatctgcacaggcgactcgacaaggttgaggaagaagttaatctccatgaggattttgtcttcataaaaaagctgaagcgatgtaataagggtgaggggtcccttagcctgcttaactgtgaagagatgagaagacagttcgaggaccttgtgaaagacatcaccctgaacaaggaggaaaagaaggagaatgacgaagatcctcaaatcgcagctcatgtagtttccgaggcaaactcaaatgctgcaagtgtacttcagtgggccaaaaaaggctattacaccatgaaatcaaatctcgtcatgcttgaaaacgggaagcagctcaccgtaaagcgagagggtctttattatgtttacacacaggtgactttctgttcaaatcgcgagccctccagccaacggccatttatagtcggactttggctgaagccttcttccggctctgagcgcatactcctgaaagccgcaaacacacatagttcttcccagctctgtgagcaacaaagcgtgcatctgggaggggttttcgagttgcaagccggtgcctccgtttttgtaaacgtgacagaagcttcccaagttatccatcgagttggattctccagcttcggccttttgaaactctga

SEQ ID NO: 65 - DNA, 1773 bp, Mus musculus
  MISC_FEATURE (1)..(1773): Mouse Codon Optimized Secreted Single-Chain IL-12
atgtgtccccaaaaactgactatatcttggttcgctattgtccttcttgtctcaccacttatggcaatgtgggaactggagaaagacgtctacgtagtggaggtggattggactcctgacgcacccggagaaacagtgaacctgacatgcgataccccagaggaagatgacataacttggaccagcgatcagagacacggagtcatcggctccgggaaaacacttactattacagtaaaagaatttctcgatgctggccaatatacatgccacaaaggtggggagactttgtcccactcccatcttctgctccataagaaggagaacggcatttggtcaaccgaaatcttgaaaaacttcaagaataaaacttttttgaaatgcgaggcccctaactacagtggccgatttacttgctcttggttggttcagcgaaacatggatcttaaattcaatataaagtcaagcagctcctctcctgactcaagagccgtaacctgtggcatggcatctttgtcagcagagaaagtaacacttgaccaacgagactacgaaaaatattcagtaagctgtcaggaagatgtgacctgtcccacagcagaagaaactctccccattgaacttgcactggaggctcggcaacaaaacaaatatgagaactatagtacctcatttttcatcagagatatcatcaagccagatccacctaagaacctgcagatgaaaccacttaaaaacagtcaagtggaagtttcatgggagtaccctgacagctggagcacaccccacagttattttagtttgaagttcttcgtccgcatccagcgaaaaaaggaaaaaatgaaagagactgaagagggatgcaatcaaaaaggtgcttttcttgtggaaaagaccagcacagaggtccaatgtaagggaggtaatgtttgcgtgcaggcacaggatcggtactataattcctcctgctctaaatgggcatgtgtgccttgccgggtgcgaagcgggggaggaggcagtggtggagggggttctgggggtggtctcgccagtggaggatctatggtgtcagtaccaaccgccagtccaagtgccagttcaagcagttcacagtgcagatcctcaatgtgtcagtcaagatacctgctgttcttggcaacccttgccctgttgaaccacctttctctggcacgagttataccagttagcgggcccgctaggtgtttgagtcagagtcgaaatcttctgaagactaccgacgacatggtaaaaaccgcccgagagaaattgaagcattatagctgcactgcagaagacatcgatcacgaggacataacccgggatcagaccagtactcttaagacatgtttgcccctcgaattgcataagaatgaatcttgccttgctaccagggaaacctcaagtaccactcggggtagctgtctccctccccagaagacttccttgatgatgacactctgtttggggtccatttatgaggacttgaagatgtatcaaactgaatttcaggcaatcaatgccgccctccaaaaccataaccatcaacagataatacttgacaagggaatgcttgtcgctatagacgaactgatgcaaagcttgaaccataacggtgaaactctgcgacagaaaccacccgttggagaggctgacccttatcgggtgaaaatgaaactctgtatactgctgcacgcttttagtactcgggtcgtgactatcaacagagttatgggttaccttagctccgcatga

SEQ ID NO: 66 - protein, 7 aa, Artificial Sequence
  Synthetic metalloproteinase cleavage misc_feature deleted from CD40L to form membrane anchored CD40L
SFEMQKG

SEQ ID NO: 67 - DNA, 2337 bp, Nipah henipavirus
  MISC_FEATURE (1)..(2337): Nucleic acid encoding Nipah F fusion protein (NC_002728.1)
aggagccaagctcttgcctcgttcagaaggttaaacaagcattcttaccattggatcaacaaaaggattggttttatcgtctaagaaatttattgaaaggcaaagaaattcctggttttatgttgaatgaggtgtatcaaactaaggagaccttctaacagccaggtcataggaatataaataaaaataagaataaaattgattccatcggaagattcatttcaagaagtgatcaaatcaaagcggttggcagacctaccaatcatataccacaagactcgacaatggtagttatacttgacaagagatgttattgtaatcttttaatattgattttgatgatctcggagtgtagtgttgggattctacattatgagaaattgagtaaaattggacttgtcaaaggagtaacaagaaaatacaagattaaaagcaatcctctcacaaaagacattgttataaaaatgattccgaatgtgtcgaacatgtctcagtgcacagggagtgtcatggaaaattataaaacacgattaaacggtatcttaacacctataaagggagcgttagagatctacaaaaacaacactcatgaccttgtcggtgatgtgagattagccggagttataatggcaggagttgctattgggattgcaaccgcagctcaaatcactgcaggtgtagcactatatgaggcaatgaagaatgctgacaacatcaacaaactcaaaagcagcattgaatcaactaatgaagctgtcgttaaacttcaagagactgcagaaaagacagtctatgtgctgactgctctacaggattacattaatactaatttagtaccgacaattgacaagataagctgcaaacagacagaactctcactagatctggcattatcaaagtacctctctgatttgctttttgtatttggccccaaccttcaagacccagtttctaattcaatgactatacaggctatatctcaggcattcggtggaaattatgaaacactgctaagaacattgggttacgctacagaagactttgatgatcttctagaaagtgacagcataacaggtcaaatcatctatgttgatctaagtagctactatataattgtcagggtttattttcctattctgactgaaattcaacaggcctatatccaagagttgttaccagtgagcttcaacaatgataattcagaatggatcagtattgtcccaaatttcatattggtaaggaatacattaatatcaaatatagagattggattttgcctaattacaaagaggagcgtgatctgcaaccaagattatgccacacctatgaccaacaacatgagagaatgtttaacgggatcgactgagaagtgtcctcgagagctggttgtttcatcacatgttcccagatttgcactatctaacggggttctgtttgccaattgcataagtgttacatgtcagtgtcaaacaacaggcagggcaatctcacaatcaggagaacaaactctgctgatgattgacaacaccacctgtcctacagccgtactcggtaatgtgattatcagcttagggaaatatctggggtcagtaaattataattctgaaggcattgctatcggtcctccagtctttacagataaagttgatatatcaagtcagatatccagcatgaatcagtccttacaacagtctaaggactatatcaaagaggctcaacgactccttgatactgttaatccatcattaataagcatgttgtctatgatcatactgtatgtattatcgatcgcatcgttgtgtatagggttgattacatttatcagttttatcattgttgagaaaaagagaaacacctacagcagattagaggataggagagtcagacctacaagcagtggggatctctactacattgggacatagtgtattcagattgatgaaattatgttagagaaatcagaaaacttctgactttcagaaatggattgtatacaattagttagatcatcctgaataatcgaggtgagaacattgcaactataaaatcagatcatgtaaatagttgtaaaaaattaaaagcttcttttaattcttttgaacaataatttaattaatatataacatattctctcacacgagcgctaacctatacactctctactaatattttatactcataattaatgatataatgacaaataaggattcaaattggattatgatatagtttcatactacaatagcatttcgaccaagaaaatatccttacaattatacaatgtacttaaccgtgaatatgtaattgataatttccctttagaaatttaataaaaaa

SEQ ID NO: 68 - protein, 546 aa, Nipah henipavirus
  MISC_FEATURE (1)..(546): Nipah F fusion protein amino acid sequence (NP_112026.1)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 69 - DNA, 2546 bp, Nipah henipavirus
  MISC_FEATURE (1)..(2546): nucleic acid Nipah G attachment glycoprotein (NC_002728.1)
aggacccaggtccataactcattggatacttaactgtatctttctaagctatcacatatcaaaggagagattgaatgcttttttggagatctagatcattactatatgtgtctcctataatcacatcataggagtgaaccataatacacatctttgggtaggggaaggaaagtattgttgacgtactgattgatctgcttgagtcaaataatcagtcataacaattcaagaaaatgccggcagaaaacaagaaagttagattcgaaaatactacttcagacaaagggaaaattcctagtaaagttattaagagctactacggaaccatggacattaagaaaataaatgaaggattattggacagcaaaatattaagtgctttcaacacagtaatagcattgcttggatctatcgtgatcatagtgatgaatataatgatcatccaaaattacacaagatcaacagacaatcaggccgtgatcaaagatgcgttgcagggtatccaacagcagatcaaagggcttgctgacaaaatcggcacagagatagggcccaaagtatcactgattgacacatccagtaccattactatcccagctaacattgggctgttaggttcaaagatcagccagtcgactgcaagtataaatgagaatgtgaatgaaaaatgcaaattcacactgcctcccttgaaaatccacgaatgtaacatttcttgtcctaacccactcccttttagagagtataggccacagacagaaggggtgagcaatctagtaggattacctaataatatttgcctgcaaaagacatctaatcagatattgaagccaaagctgatttcatacactttacccgtagtcggtcaaagtggtacctgtatcacagacccattgctggctatggacgagggctattttgcatatagccacctggaaagaatcggatcatgttcaagaggggtctccaaacaaagaataataggagttggagaggtactagacagaggtgatgaagttccttctttatttatgaccaatgtctggaccccaccaaatccaaacaccgtttaccactgtagtgctgtatacaacaatgaattctattatgtactttgtgcagtgtcaactgttggagaccctattctgaatagcacctactggtccggatctctaatgatgacccgtctagctgtgaaacccaagagtaatggtgggggttacaatcaacatcaacttgccctacgaagtatcgagaaagggaggtatgataaagttatgccgtatggaccttcaggcatcaaacagggtgacaccctgtattttcctgctgtaggatttttggtcaggacagagtttaaatacaatgattcaaattgtcccatcacgaagtgtcaatacagtaaacctgaaaattgcaggctatctatggggattagaccaaacagccattatatccttcgatctggactattaaaatacaatctatcagatggggagaaccccaaagttgtattcattgaaatatctgatcaaagattatctattggatctcctagcaaaatctatgattctttgggtcaacctgttttctaccaagcgtcattttcatgggatactatgattaaatttggagatgttctaacagtcaaccctctggttgtcaattggcgtaataacacggtaatatcaagacccgggcaatcacaatgccctagattcaatacatgtccagagatctgctgggaaggagtttataatgatgcattcctaattgacagaatcaattggataagcgcgggtgtattccttgacagcaatcagaccgcagaaaatcctgtttttactgtattcaaagataatgaaatactttatagggcacaactggcttctgaggacaccaatgcacaaaaaacaataactaattgttttctcttgaagaataagatttggtgcatatcattggttgagatatatgacacaggagacaatgtcataagacccaaactattcgcggttaagataccagagcaatgtacataaaaatcaacctcataatttaatggattgatctaatataatgataataatcgtacaaagacatgtgatgtaaacaaaattgttgtaattaaataagtcctcagctgaatacttttttaagattagcaatagcatgtttttccagttattggatagttgataatataattctgaaactgggttaataaataatcttgatcggtgatctttgagaacaatgatatcatatagttcatcaagtgataatcaattctttatatgtacactttagagtatattttgagacttagtattttcggcccgaatgttaaatttaatagttcatacataacctaaactcaagttctaagcataatgataacaattaatgcgaacttgtcttgatgtaaggaagatttgatattaactgagactccacttgatatagtagagctgaatcttgtaaataaattataatgaatagtttattcaaagattatcattcatattagtgtaaattaagaaaaa

SEQ ID NO: 70 - protein, 602 aa, Nipah henipavirus
  MISC_FEATURE (1)..(602): Nipah G attachment glycoprotein amino acid sequence(NP_112027.1)
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 71 - DNA, 13616 bp, Artificial Sequence
  Synthetic VSV, codon optimized Lassa glycoprotein, and two murine codon optimized transgenes CD40L and IL12
acgaagacaaacaaaccattattatcattaaaaggctcaggagaaactttaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgtcactacgcgtccaccatgggtcagattgtgacattcttccaggaagtgcctcatgtgattgaagaggtgatgaacattgtgctcattgccctgtccgtgctcgccgtgctgaaaggtctgtacaattttgccacctgtggcctggtgggtctggtgacattcctcctgctgtgtggtaggtcctgcacaaccagcctctacaaaggtgtgtacgagctccagacactggaactgaacatggagacactcaatatgaccatgcctctctcctgcacaaagaacaacagccatcattacatcatggtgggcaatgagacaggcctggaactgaccctgaccaacacaagcattattaatcacaaattttgcaatctgtccgatgcccacaaaaagaacctctacgaccacgccctcatgagcattatctccaccttccacctgtccatccccaacttcaatcagtacgaggccatgagctgcgattttaatggcggtaagattagcgtgcagtacaacctgagccacagctacgccggcgatgccgccaaccattgtggtacagtggccaatggtgtgctgcagacctttatgaggatggcctggggtggtagctacattgccctcgactccggcaggggcaactgggactgtattatgaccagctaccagtacctgattatccagaatacaacctgggaagatcactgccagttctccagaccatcccccatcggttacctcggtctcctctcccagaggaccagagatatttacattagcagaagactgctcggcacattcacatggacactgtccgattccgaaggtaaagacacaccaggtggctactgtctgaccaggtggatgctcattgaggccgaactcaaatgcttcggtaacacagccgtggccaaatgtaatgagaagcatgatgaggaattttgtgacatgctgaggctgtttgacttcaacaaacaggccattcagaggctgaaagccgaagcccagatgagcattcagctgatcaacaaagccgtgaacgccctgattaatgaccagctcatcatgaagaaccatctgagggacatcatgggcattccatactgtaattacagcaagtactggtatctcaaccacacaaccacaggtagaacatccctgcccaaatgttggctcgtgtccaatggttcctacctgaacgagacccacttttccgatgatattgaacagcaggccgacaatatgatcaccgagatgctgcagaaggagtacatggagaggcagggtaagacaccactgggtctggtggacctctttgtgttcagcacaagcttctacctcattagcatcttcctccacctggtgaaaattccaacacataggcatattgtgggcaagtcctgtcccaaacctcacagactgaatcatatgggcatttgttcctgtggtctctacaaacagcctggtgtgcctgtgaaatggaagagatgagctagcctcaaatcctgctaggtatgaaaaaaactaacagatatcacgctcgagactggtaccccaccatgatcgaaacttacagccagccaagccccaggtccgtcgccactggccttcctgcgtccatgaagatctttatgtacctcttgacggtattcctcatcacccaaatgatcgggtccgttctgttcgccgtgtacttgcatcgcagattggacaaggtggaggaagaggtgaacctgcacgaagacttcgtcttcatcaaaaagctgaagcgctgtaataagggtgagggaagcctctctctgcttaactgcgaagagatgcgccggcagttcgaggacttggtgaaggatatcactttgaacaaggaagagaaaaaggagaactcctttgagatgcaacggggcgacgaggaccctcagattgcagcccacgtggtctctgaggccaactctaacgcagcctccgtgcttcagtgggctaaaaagggctattacacaatgaaaagtaacctcgtcatgctggagaacgggaagcagctgaccgtgaaaagagagggcctctattacgtgtacacgcaggtgacgttctgcagcaatcgtgagccctcatcccagcgtccattcatcgtgggcctctggcttaaacctagctcaggatccgagaggatcctccttaaggcggctaacacacactcctcttcccaattgtgcgagcaacagtctgtacacctgggtggcgtgttcgagctgcaggctggcgcgtctgtctttgtgaatgttaccgaggcatctcaggtgattcaccgcgtgggcttctccagttttggactcctgaagctgtagcgtacgtatgaaaaaaactaacagatatcctcgagccaccatgtgtcctcagaagctgacaatttcttggttcgccatcgtgctcttggtttctccactgatggctatgtgggagcttgaaaaggacgtgtacgtggtcgaggtggactggaccccagatgcgcctggcgagacggtgaatttgacatgtgatacccctgaagaggacgatattacatggacttctgatcaaaggcacggcgtgatcggcagtggtaaaacactgacaattaccgtgaaggagttcctcgacgccggccaatatacctgtcacaaaggtggcgaaaccctgtcccattcccacctgctccttcacaaaaaggagaacggtatctggtccacggagattctcaaaaactttaagaacaagacattcctgaaatgcgaggcccccaactactcaggcagattcacatgttcttggctggtgcagcgcaacatggacttgaagttcaacattaagagctcctctagctcccctgactctcgtgctgtgacctgtggtatggccagcctgtcagccgaaaaagttaccctggaccagcgcgactacgagaagtattccgtgtcttgtcaagaggatgttacatgtcccactgccgaggaaacgctgccaatcgagctggcattggaggctcgccaacagaacaagtacgagaattactccacatcctttttcatccgcgacatcattaaaccagatcctcccaaaaacctgcagatgaagcccctgaagaacagtcaggtcgaggtgagctgggagtatcccgacagttggagcaccccacattcctacttctccctcaagttctttgttcggatccagagaaaaaaggagaaaatgaaggagaccgaagagggttgcaaccagaagggcgctttcctggtggagaagacttccactgaagtgcagtgcaaagggggaaacgtttgcgtacaggcccaagaccgctattacaactcttcctgtagtaagtgggcatgtgtgccttgtagagtgaggtcagttcctggagtaggggtacctggagtgggccgcgtgattcctgtttcaggcccggcgcgttgtctctcccagagccgcaaccttctgaagaccacagacgatatggtcaaaaccgctagggagaagctgaaacactatagctgcactgccgaggacattgaccacgaggacatcactcgtgaccagacatctaccctgaagacttgcctccctctggagcttcacaagaatgagagctgcctggccactcgtgagacttcatctactacccgcggcagctgtctccctccacagaagacctccctgatgatgactctgtgtctgggctccatctatgaggacctgaagatgtaccagacggagttccaggccatcaacgctgcattgcagaaccacaaccatcagcaaattatcctggataagggcatgctggtggctattgatgagctcatgcagtctctcaaccacaacggtgagaccctgcgtcaaaagccacctgtgggcgaggccgacccgtatcgggtgaagatgaaactctgcatccttctgcatgccttcagcaccagggtggtcacgatcaacagggtgatgggctatctgagctccgcctaacctagggctagccagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 72 - protein, 15 aa, Artificial Sequence
  Synthetic (G4S)3 Linker sequence
GGGGSGGGGSGGGGS

SEQ ID NO: 73 - protein, 19 aa, Artificial Sequence
  Synthetic (G4S)2GGGLASGGS Linker sequence
GGGGSGGGGSGGGLASGGS

SEQ ID NO: 74 - protein, 10 aa, Artificial Sequence
  Synthetic Linker sequence
VPGVGVPGVG

SEQ ID NO: 75 - DNA, 18 bp, Artificial Sequence
  Synthetic exemplary minimal transcriptional STOP-START sequence
tatcaaaaaaactaacag

SEQ ID NO: 76 - DNA, 40 bp, Artificial Sequence
  Synthetic longer SS sequence
cgtacgtatgaaaaaaactaacagatatcctcgagccacc

SEQ ID NO: 77 - DNA, 18 bp, Artificial Sequence
  Synthetic minimal Stop-Start sequence
tatgaaaaaaactaacag

SEQ ID NO: 78 - DNA, 13581 bp, Artificial Sequence
  Synthetic Full-length sequence that consists of the VSV backbone from FJ478454 containing the chimeric cassette in place of VSV G. This cassette consists of mammalian codon optimized Lassa G, human CD40L and human IL12.
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctactctcgtctagatcaggcggtaggagagatagatgagatcgaagcacaacgagctgaaaagtccaattatgagttgttccaagaggacggagtggaagagcatactaggccctcttattttcaggcagcagatgattctgacacagaatctgaaccagaaattgaagacaatcaaggcttgtatgtaccagatccggaagctgagcaagttgaaggctttatacaggggcctttagatgactatgcagatgaggacgtggatgttgtattcacttcggactggaaacagcctgagcttgaatccgacgagcatggaaagaccttacggttgacattgccagagggtttaagtggagagcagaaatcccagtggcttttgacgattaaagcagtcgttcaaagtgccaaacactggaatctggcagagtgcacatttgaagcatcgggagaaggggtcatcataaaaaagcgccagataactccggatgtatataaggtcactccagtgatgaacacacatccgtaccaatcagaagccgtatcagatgtttggtctctctcaaagacatccatgactttccaacccaagaaagcaagtcttcagcctctcaccatatccttggatgaattgttctcatctagaggagaattcatctctgtcggaggtaacggacgaatgtctcataaagaggccatcctgctcggtctgaggtacaaaaagttgtacaatcaggcgagagtcaaatattctctgtagactatgaaaaaaagtaacagatatcacaatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgttgacactatgggtcagattgtgacattcttccaggaagtgcctcatgtgattgaagaggtgatgaacattgtgctcattgccctgtccgtgctcgccgtgctgaaaggtctgtacaattttgccacctgtggcctggtgggtctggtgacattcctcctgctgtgtggtaggtcctgcacaaccagcctctacaaaggtgtgtacgagctccagacactggaactgaacatggagacactcaatatgaccatgcctctctcctgcacaaagaacaacagccatcattacatcatggtgggcaatgagacaggcctggaactgaccctgaccaacacaagcattattaatcacaaattttgcaatctgtccgatgcccacaaaaagaacctctacgaccacgccctcatgagcattatctccaccttccacctgtccatccccaacttcaatcagtacgaggccatgagctgcgattttaatggcggtaagattagcgtgcagtacaacctgagccacagctacgccggcgatgccgccaaccattgtggtacagtggccaatggtgtgctgcagacctttatgaggatggcctggggtggtagctacattgccctcgactccggcaggggcaactgggactgtattatgaccagctaccagtacctgattatccagaatacaacctgggaagatcactgccagttctccagaccatcccccatcggttacctcggtctcctctcccagaggaccagagatatttacattagcagaagactgctcggcacattcacatggacactgtccgattccgaaggtaaagacacaccaggtggctactgtctgaccaggtggatgctcattgaggccgaactcaaatgcttcggtaacacagccgtggccaaatgtaatgagaagcatgatgaggaattttgtgacatgctgaggctgtttgacttcaacaaacaggccattcagaggctgaaagccgaagcccagatgagcattcagctgatcaacaaagccgtgaacgccctgattaatgaccagctcatcatgaagaaccatctgagggacatcatgggcattccatactgtaattacagcaagtactggtatctcaaccacacaaccacaggtagaacatccctgcccaaatgttggctcgtgtccaatggttcctacctgaacgagacccacttttccgatgatattgaacagcaggccgacaatatgatcaccgagatgctgcagaaggagtacatggagaggcagggtaagacaccactgggtctggtggacctctttgtgttcagcacaagcttctacctcattagcatcttcctccacctggtgaaaattccaacacataggcatattgtgggcaagtcctgtcccaaacctcacagactgaatcatatgggcatttgttcctgtggtctctacaaacagcctggtgtgcctgtgaaatggaagagatgatgagctagccagattcttcatgtttggaccaaatcaacttgtgataccatgctcaaagaggcctcaattatatttgagtttttaatttttatgaaaaaaactaacagcaatcggcgcgtggtaccccaccatgatcgagacatataaccagacttctccgcgcagcgctgccacgggtttgcccatcagcatgaagatcttcatgtatctcctgaccgtgttcctgatcacccaaatgatcgggtccgcacttttcgcggtctacctccaccggcgcctggacaaaattgaagatgagcggaacctgcacgaggatttcgtgttcatgaaaaccatccagcgctgcaacaccggcgaacgctcactctcccttctgaactgcgaagagatcaagtcccaattcgagggattcgttaaggatatcatgctgaacaaggaagagaccaaaaaggaaaacagttttgaaatgcagaagggtgatcagaacccacagattgcagctcatgtcatcagcgaagcttccagcaaaacaacctcagtgctgcagtgggccgagaagggctattacactatgtctaataacctggtgacactggagaatggtaagcagttgacagtcaagcgtcagggattgtactatatctacgcccaggtgacgttttgctccaatcgcgaggctagttctcaggctccgttcatcgcgagcctgtgtctgaagagccccggccggttcgaacgcatccttctgagggctgcgaatacacactcctcagccaagccctgcggacaacagagtattcacctcggaggggtcttcgaactgcagcctggggcttccgtgtttgtgaatgtcaccgacccctcccaggtcagtcacggaactggtttcacgagcttcggactcctgaaactctgacgtacgtatgaaaaaaactaacagatatcctcgagccaccatgtgtcaccaacagctcgtcatcagctggttctccctcgtgttcctggcctcccctctggtggcgatctgggaattgaaaaaggatgtgtacgttgtggagctggactggtatcccgacgccccaggtgaaatggtagtgctcacctgtgacacaccagaagaggacgggattacatggacactggaccaatcctctgaggttctggggtccggtaagaccttgaccatccaagtcaaagagttcggcgacgctggccagtacacatgccacaagggcggtgaagtccttagccattctctgcttctgctccacaagaaagaagacggaatttggtctaccgacatcctgaaggaccagaaggagcctaagaacaagacatttcttaggtgtgaagccaagaactactccggccgcttcacctgttggtggctgacaaccatttctaccgatctgaccttcagtgtgaaatctagccgtggcagttccgatccgcagggggttacatgtggagctgccacactgtccgccgagagggtacgtggcgacaacaaggagtatgaatactctgtggagtgccaggaagactctgcttgccctgcagctgaagagagtctgcccatcgaggtgatggtcgacgctgtgcacaagcttaagtacgagaactacaccagctccttctttattcgcgacattatcaagcccgaccctcccaagaacctgcaactcaagccactgaagaactccagacaggtggaggtgtcatgggaatacccagatacctggagcaccccacacagctacttctcactgaccttctgcgttcaggtacagggtaagtcaaagcgtgagaaaaaggaccgcgtgttcaccgacaaaacttccgcaaccgtcatctgccggaagaacgctagcatctctgtacgggctcaagacagatattactcctcatcctggtccgagtgggcgtccgtgccatgttccgttcctggagtaggggtacctggagtgggcgcccgcaatttgccagttgccacaccggaccccggcatgtttccctgcctgcatcactcccagaacctcttgcgcgccgtgtccaatatgctgcagaaggcacgccaaaccctggagttctacccctgcaccagcgaggaaatcgaccacgaggacatcaccaaggataaaacctcaactgtcgaagcttgtctgcccctggaactgactaagaacgagtcctgcctgaactcccgtgaaacctctttcatcaccaatggctcttgcctcgcttccaggaagacctccttcatgatggccctgtgtctgtcttccatctatgaggacctcaaaatgtaccaggttgagttcaagaccatgaacgccaaactgctcatggaccctaagcgtcagatctttttggatcagaacatgctggcagtcattgacgaactgatgcaagctctgaactttaatagtgaaaccgtaccccagaagtccagcctggaagagcctgacttctacaaaaccaaaatcaagctgtgtattctgcttcacgcgttccgtattagggcggtgaccatcgatcgcgtgatgtcttacctgaacgcaagctaacctaggtatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttggcagttatccagcaatcatttccgattaggtgggaggctatttcagtaaaaggaggatacaagcagaagtggagtactagaggtgatgggctcccaaaagatacccgaatttcagactccttggccccaatcgggaactggatcagatctttggaattggtccgaaaccaagttcgtctaaatccattcaataagatcttgttcaatcagctatgtcgtacagtggataatcatttgaagtggtcaaatttgcgaaaaaacacaggaatgattgaatggatcaatgggcgaatttcaaaagaagaccggtctatactgatgttgaagagtgacctacatgaggaaaactcttggagagattaaaaaatcatgaggagactccaaactttaagtatgaaaaaaactttgatccttaagaccctcttgtggtttttattttttatctggttttgtggtcttcgt

(19) United States
(12) Patent Application Publication
(10) Pub. No.: US 2024/0358780 A1
(43) Pub. Date: Oct. 31, 2024

(54) ARMED CHIMERIC ONCOLYTIC VIRUSES

(71) Applicant: Implicyte, Inc., Haddonfield, NJ (US)

(72) Inventors: Lutz B Giebel, Oro Valley, AZ (US); Matthew H Stremlau, Washington, DC (US); John S Swartley, Haddonfield, NJ (US)

(73) Assignee: Implicyte, Inc., Haddonfield, NJ (US)

(21) Appl. No.: 18/553,632

(22) PCT Filed: Apr. 3, 2022
(86) PCT No.: PCT/US2022/023223

§ 371 Date: Oct. 2, 2023

Related U.S. Application Data
(60) Provisional application No. 63/171,028, filed on Apr. 5, 2021.
(60) Provisional application No. 63/295,910, filed on Jan. 2, 2022.

(51) Int. Cl. A61K 35/766; A61P 35/00; A61P 37/04; C07K 14/54; C07K 14/705; C12N 7/00

(52) U.S. Cl. CPC A61K 35/766; A61P 35/00; A61P 37/04; C07K 14/5434; C07K 14/70578; C12N 7/00; C12N 2760/20222; C12N 2760/20232; C12N 2760/20243; C12N 2760/20252; C12N 2760/20271

(57) ABSTRACT

The present invention relates generally to the field of cancer therapy and oncolytic viruses. More particularly, it concerns armed, chimeric oncolytic viruses.

### CROSS-REFERENCE

This application claims the benefit of U.S. Provisional Patent Application Ser. No. 63/171,028, filed Apr. 5, 2021, entitled “ARMED CHIMERIC ONCOLYTIC VIRUSES” and U.S. Provisional Patent Application Ser. No. 63/295,910, filed Jan. 2, 2022, entitled “ARMED CHIMERIC ONCOLYTIC VIRUSES”, both of which are expressly incorporated herein by reference.

### REFERENCE TO SEQUENCE LISTING

The sequence included herewith is expressly incorporated into this specification.

### BACKGROUND

### Field of the Invention

The present invention relates generally to the field of cancer therapy and oncolytic viruses. More particularly, it concerns armed, chimeric oncolytic viruses.

### Description of Related Art

Vesicular stomatitis virus (VSV) is an enveloped, negative-sense, single-strand RNA virus in the Rhabdoviridae family. In recent years, recombinant altered versions of VSV have shown considerable potential as the molecular basis for live vaccines engineered to express antigenic proteins from other viruses (Kurup, et al., J. Virol., 89:144-154 (2015); Clarke, et al., Springer Semin. Immunopathol., 28:239-253 (2006); Geisbert, et al., PloS Pathog., 4:e1000225 (2008); Geisbert, et al., J. Virol., 83:7296-7304 (2009)). VSV has also shown promise as an oncolytic virus (Wongthida, et al., Hum. Gene. Ther., 22:1343-1353 (2011); Obuchi, et al., J. Virol., 77:8843-8856 (2003); Ozduman, et al., J. Virol., 83:11540-11549 (2008); van den Pol, et al., J. Virol., 87:1019-1034 (2013); Wollmann, et al., J. Virol., 79:6005-6022 (2005)).

### SUMMARY

In one embodiment the disclosure provides an armed oncolytic virus comprising a chimeric Vesicular Stomatitis Virus (VSV), wherein the chimeric VSV comprises a VSV background and a heterologous viral glycoprotein selected from the group consisting of an Arenaviridae family virus, a Filovirus family virus, a Togovirus family virus, and a Paramyxoviridae family virus and a heterologous immunomodulatory molecule. More particularly the armed oncolytic virus comprises a heterologous glycoprotein selected from the group consisting of Lassa virus glycoprotein, an Ebola virus glycoprotein, a Chikungunya virus glycoprotein, and functional fragment or fragments thereof, in place of the VSV G-protein. In certain embodiments the armed oncolytic virus comprises an immunomodulatory molecule that may be a secreted single chain IL-12, membrane anchored IL-12, secreted CD40L and/or membrane anchored CD40L. The IL-12 molecule may be a secreted or membrane anchored and may be encoded as a single chain molecule fusion protein of p35 and p40 subunits fused by a linker. In some embodiments the membrane anchored IL-12 comprises a transmembrane domain that may be the transmembrane domain from CD28, CD8 or B7.1 transmembrane domains. In some embodiments when the armed oncolytic virus comprises membrane anchored CD40L, the CD40L lacks the endogenous metalloprotease cleavage site. In some embodiments when the comprises IL-12, it is not a wild type IL-12.

In one embodiment, the disclosure provides a method of treating cancer in a subject in need thereof comprising administering to said subject an armed oncolytic virus as described herein.

It is contemplated that any embodiment of a method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 shows an overview of an ARCH VSV platform. N, P, M, G and L refer to coding sequences for VSV proteins. Specifically, the proteins are nucleocapsid (N), phosphoprotein (P), matrix (M) protein, glycoprotein (G) and large (L) viral polymerase. The VSV G protein is replaced with a heterologous G protein from a different virus, such as Ebola (E), Lassa (L) or Chikungunya (G). At least two transgenes (TG1 and TG2) may be inserted into the viral genome aided by Restriction Endonuclease cleavage sites (RE). Exemplary linkers include GGGGS (SEQ ID NO:14), (G₄S)₃ (SEQ ID NO:72), (G₄S)₂GGGLASGGS (SEQ ID NO:73), and VPGVGVPGVG (SEQ ID NO:74).

FIG. 2 depicts the genomic organization of single-chain IL-12 and membrane anchored single chain IL-12.

FIG. 3 depicts the genomic organization of wild-type CD40L and membrane anchored CD40L with a deletion of nucleic acid bases 328-349 (encoding amino acids 100-116).

FIG. 4 shows a family tree of the Arenaviridae family of viruses.

FIG. 5 shows a family tree of the Filovirus family of viruses.

FIG. 6 shows a family tree of the Alphavirus genus of viruses.

FIG. 7 shows an overview of an ARCH VSV platform. N, P, M, G and L refer to coding sequences for VSV proteins. The VSV G protein is replaced with at least one or two heterologous G protein from a different virus or viruses, such as Ebola (E), Lassa (L) or Chikungunya (G). At least two transgenes (TG1 and TG2) may be inserted into the viral genome aided by Restriction Endonuclease cleavage sites (RE).

FIG. 8 shows a family tree of the Paramyxoviridae family of viruses.

FIG. 9 shows an overview of alternative configurations an ARCH VSV platform. N, P, M, G and L refer to coding sequences for VSV proteins. The VSV G protein is replaced with a heterologous G protein from a different virus, such as Ebola (E), Lassa (L) or Chikungunya (G). At least two transgenes (TG1 and TG2) may be inserted into the viral genome aided by Restriction Endonuclease cleavage sites (RE). The transgenes may flank the G protein or in some embodiments the transgenes may be 5′ or 3′ of the G protein. SS depicts an exemplary minimal transcriptional STOP-START sequence (SEQ ID NO:75). A longer SS is depicted as well that includes restriction endonuclease sites, start and stop sequences as well as a Kozak sequence (SEQ ID NO:76).

FIG. 10 depicts an exemplary configuration of a chimeric VSV construct. The Chimeric Cassette is inserted in place of the wild-type VSV glycoprotein (and between two restriction sites, MluI and AvrII). Minimal Stop-Start sequences are indicated and have the following sequences: TATGAAAAAAACTAACAG (SEQ ID NO:77).

### DESCRIPTION

VSV have been examined as a delivery vehicle for vaccines or expression of other heterologous proteins in humans. In addition, VSV, including chimeric VSV have been examined as oncolytic viruses and have achieved some successes in animal models. However, the immune response provoked by these VSV remains inconsistent and sub-optimal for cancer therapy. Accordingly, there exists a need in the art for improved VSV-based oncolytic viruses. As such, the present disclosure provides improved chimeric VSV viruses comprising heterologous immunomodulatory molecules. These Armed, Chimeric VSV may be referred to herein as ARCH VSV.

Therefore, it is an object of the invention to provide recombinant oncolytic viruses, preferably with improved safety and superior cytolytic profiles.

It is a further object of the invention to provide pharmaceutical compositions including an effective amount of recombinant oncolytic viruses to treat cancer in a human subject.

It is another object of the invention to provide methods of using recombinant oncolytic virus to kill cancer cells.

It is a further object of the invention to increase the body's immune response against cancer cells.

As used herein, the term “isolated” describes a compound of interest (e.g., either a polynucleotide or a polypeptide) that is in an environment different from that in which the compound naturally occurs e.g., separated from its natural milieu such as by concentrating a peptide to a concentration at which it is not found in nature. “Isolated” includes compounds that are within samples that are substantially enriched for the compound of interest and/or in which the compound of interest is partially or substantially purified. With respect to nucleic acids, the term “isolated” includes any non-naturally-occurring nucleic acid sequence, since such non-naturally-occurring sequences are not found in nature and do not have immediately contiguous sequences in a naturally-occurring genome.

As used herein, the term “nucleic acid(s)” refers to any nucleic acid containing molecule, including, but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. In accordance with standard nomenclature, nucleic acid sequences are denominated by either a three letter, or single letter code as indicated as follows: adenine (Ade, A), thymine (Thy, T), guanine (Gua, G) cytosine (Cyt, C), uracil (Ura, U).

As used herein, the term “polynucleotide” refers to a chain of nucleotides of any length, regardless of modification (e.g., methylation).

As used herein, the term “gene” refers to a nucleic acid (e.g., DNA or RNA) sequence that comprises coding sequences necessary for the production of a polypeptide, RNA (e.g., including but not limited to, mRNA, tRNA and rRNA) or precursor. The polypeptide, RNA, or precursor can be encoded by a full-length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “nucleic acid molecule encoding,” refers to the order or sequence of nucleotides along a strand of nucleotides. The order of these nucleotides determines the order of amino acids along the polypeptide (protein) chain. The nucleotide sequence thus codes for the amino acid sequence.

As used herein, the term “polypeptide” refers to a chain of amino acids of any length, regardless of modification (e.g., phosphorylation or glycosylation). In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine. (Cys, C), Glutamine (Gin, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, a “variant,” “mutant,” or “mutated” polynucleotide contains at least one polynucleotide sequence alteration as compared to the polynucleotide sequence of the corresponding wild-type or parent polynucleotide. Mutations may be natural, deliberate, or accidental. Mutations include substitutions, deletions, and insertions.

As used herein, a “nucleic acid sequence alteration” can be, for example, a substitution, a deletion, or an insertion of one or more nucleotides. An “amino acid sequence alteration” can be, for example, a substitution, a deletion, or an insertion of one or more amino acids.

As used herein, the term “immunizing virus” includes infectious virus, viral subunits, viral proteins and antigenic fragments thereof, nucleic acids encoding viral subunits, antigenic proteins or polypeptides, and expression vectors containing the nucleic acids.

As used herein, a “vector” is a replicon, such as a plasmid, phage, or cosmid, into which another DNA segment may be inserted so as to bring about the replication of the inserted segment. The vectors described herein can be expression vectors.

As used herein, the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being effected.

As used herein, the, terms “neoplastic cells,” “neoplasia,” “tumor,” “tumor cells,” “cancer” and “cancer cells,” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign.

As used herein, an “immunogen” or “immunogenic amount” refers to the ability of a substance (antigen) to induce an immune response. An immune response is an alteration in the reactivity of an organisms' immune system in response to an antigen. In vertebrates this may involve antibody production, induction of cell-mediated immunity, complement activation or development of immunological tolerance.

As used herein, an “adjuvant” is a substance that increases the ability of an antigen to stimulate the immune system.

As used herein, “attenuated” refers to refers to procedures that weaken an agent of disease (a pathogen). An attenuated virus is a weakened, less vigorous virus. A vaccine against a viral disease can be made from an attenuated, less virulent strain of the virus, a virus capable of stimulating an immune response and creating immunity but not causing illness or less severe illness. Attenuation can be achieved by chemical treatment of the pathogen, through radiation, or by genetic modification, using methods known to those skilled in the art. Attenuation may result in decreased proliferation, attachment to host cells, or decreased production or strength of toxins.

As used herein, “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. A subject can include a control subject or a test subject.

As used herein, “identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up, to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, “treatment” or “treating” means to administer a composition to a subject or a system with an undesired condition. The condition can include a disease. “Prevention” or “preventing” means to administer a composition to a subject or a system at risk for the condition. The condition can include a predisposition to a disease. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur. It is understood that where treat or prevent are used, unless specifically indicated otherwise, the use of the other word is also expressly disclosed.

### Compositions

VSV, a member of the Rhabdoviridae family, is enveloped and has a negative-strand 11.2-kb RNA genome that comprises five protein-encoding genes (N, P, M, G, and L) (Lyles, et al., Fields virology, 5th ed., Lippincott Williams & Wilkins, 1363-1408 (2007)). It is a nonhuman pathogen which can cause mild disease in livestock. Infection in humans is rare and usually asymptomatic, with sporadic cases of mild flu-like symptoms. VSV has a short replication cycle, which starts with attachment of the viral glycoprotein spikes (G) to an unknown but ubiquitous cell membrane receptor. Nonspecific electrostatic interactions have also been proposed to facilitate viral binding (Lyles, et al., Fields virology, 5th ed., Lippincott Williams & Wilkins, 1363-1408 (2007)). Upon internalization by clathrin-dependent endocytosis, the virus-containing endosome acidifies, triggering fusion of the viral membrane with the endosomal membrane. This leads to release of the viral nucleocapsid (N) and viral RNA polymerase complex (P and L) into the cytosol.

The viral polymerase initiates gene transcription at the 3′ end of the non-segmented genome, starting with expression of the first VSV gene (N). This is followed by sequential gene transcription, creating a gradient, with upstream genes expressed more strongly than downstream genes. Newly produced VSV glycoproteins are incorporated into the cellular membrane with a large extracellular domain, a 20 amino acid trans-membrane domain, and a cytoplasmic tail consisting of 29 amino acids. Trimers of G protein accumulate in plasma membrane microdomains, several of which congregate to form viral budding sites at the membrane (Lyles, et al., Fields virology, 5th ed., Lippincott Williams & Wilkins, 1363-1408 (2007)). Most cells activate antiviral defense cascades upon viral entry, transcription, and replication, which in turn are counteracted by VSV matrix protein (M). VSV M protein's multitude of functions include virus assembly by linking the nucleocapsid with the envelope membrane, induction of cytopathic effects and apoptosis, inhibition of cellular gene transcription, and blocking of host cell nucleocytoplasmic RNA transfer, which includes blocking of antiviral cellular responses (Ahmed, et al., Virology, 237:378-388 (1997)).

Certain native, engineered, and recombinant VSV strains have been shown to target several tumor types, including gliomas, and give a strong oncolytic action, both in vitro and in vivo (Paglino and van den Pol, 2011) (Wollmann, et al, 2005; 2007; 2010; Ozduman et al, 2008). However, there remains a need for improved recombinant VSVs that are both efficacious for treating cancer and exhibit low pathogenicity to healthy host cells. This is particularly important in the brain where mature neurons do not replicate, and once lost, are normally not replaced. Although some evidence indicates that attenuated VSVs show reduced neurotoxicity, CNS complications have been difficult to eliminate completely (Obuchi et al, 2003; van den Pol et al, 2002; 2009).

It has been discovered that recombinant, chimeric VSV viruses where the G gene is substituted with a gene encoding a heterologous glycoprotein protein have oncolytic potential in targeting and destroying cancer cells with little pathogenicity to healthy host cells. Recombinant VSV viruses, pharmaceutical compositions including recombinant VSV viruses, and methods of use thereof for treating cancer are provided. As discussed in more detail below, preferably, the virus targets and kills tumor cells, and shows little or no infection of normal cells.

### Chimeric G-Gene Substituted VSV Virus

The disclosed viruses are chimeric VSV viruses that are typically based on a VSV background strain, also referred to herein as a VSV backbone, wherein the G gene is substituted by a gene encoding a heterologous glycoprotein or multiple heterologous glycoproteins. As discussed in more detail below, the chimeric virus can also include additional genetic changes (e.g., additions, deletions, substitutions) relative to the background VSV virus, and can have one or more additional transgenes.

### VSV Background Strain

Useful VSV virus background strains can be viruses that are known in the art, or they can be mutants or variants of known viruses. Any suitable VSV strain or serotype may be used, including, but not limited to, VSV Indiana, VSV New Jersey, VSV Alagoas, (formerly Indiana 3), VSV Cocal (formerly Indiana 2), VSV Chandipura, VSV Isfahan, VSV San Juan, VSV Orsay, or VSV Glasgow. The VSV virus background can be a naturally occurring virus, or a virus modified, for example, to increase or decrease the virulence of the virus, and/or increase the specificity or infectivity of the virus compared to the parental strain or serotype. The virus can be a recombinant virus that includes genes from two or more strains or serotypes. For example, the VSV background strain can be a recombinant VSV with all five genes of the Indiana serotype of VSV. In another exemplary embodiments, the N, P, M, and L genes originates from the San Juan strain, and the G gene from the Orsay strain.

It may be desirable to further reduce the neurovirulence of the viruses used in the disclosed methods, particularly the virulence of the therapeutic virus, by using an attenuated virus. A number of suitable VSV mutants have been described, see for example (Clarke, et al., J. Virol., 81:2056-64 (2007), Flanagan, et al., J. Virol., 77:5740-5748 (2003), Johnson, et al., Virology, 360:36-49 (2007), Simon, et al., J. Virol., 81:2078-82 (2007), Stojdl, et al., Cancer Cell, 4:263-275 (2003)), Wollmann, et al., J. Virol., 84(3):1563-73 (2010) (epub 2010), WO 2010/080909, U.S. Published Application No. 2007/0218078, and U.S. Published Application No 2009/0175906.

Recombinant VSVs derived from DNA plasmids also typically show weakened virulence (Rose, et al., Cell, 106:539-549 (2001)). Attenuation of VSV virulence can also be accomplished by one or more nucleotide sequence alterations that result in substitution, deletion, or insertion of one or more amino acids of the polypeptide it encodes.

In some embodiments, the VSV background strain is a VSV virus modified to attenuate virus growth or pathogenicity or to reduce the ability to make infectious progeny viruses. VSV strains and methods of making such VSV strains are known in the art, and described in, for example, U.S. Published Application No. 2012/0171246. In one embodiment the VSV backbone is based on Genbank sequence FJ478454 from Lawson, N. D., Stillman, E. A., Whitt, M. A., Rose, J. K., 1995. Recombinant vesicular stomatitis viruses from DNA. Proc. Natl. Acad. Sci. U.S.A. 92, 4477-4481, which is incorporated herein by reference. In some embodiments the VSV backbone is based on Genbank sequence FJ478454 and is modified to contain sequences corresponding to the P, M and L genes from MN153298.1 (incorporated herein by reference) (i.e. the sequence for P 1396-2193 are from the corresponding sequence in MN153298.1; the sequence for M 2250-2939 is from MN153298.1; the sequence for L 7153-13,482 is from MN153298.1).

In some embodiments the VSV background strain is from NC_001560.1, FJ478454, J02428.1, EF197793, MN153298.1, all of which are expressly incorporated herein by reference. All of these sequences are variations of VSV Indiana and are available in GenBank.

For example, one strategy is to attenuate viral pathogenicity by reducing the ability of the virus to suppress host innate immune responses without compromising the yield of infectious progeny. This can be accomplished by mutating the M protein as described, for example, in Ahmed, J Virol., 82(18):9273-9277 (2008). The M protein is a multifunctional protein that is involved in the shutoff of host transcription, nuclear cytoplasmic transport, and translation during virus infection (Lyles, Microbial. Mol. Biol. Rev. 64:709-724 (2000)). Mutation and/or deletion of one or more amino acids from the M protein, for example MA51, or M51A mutants can result in viral protein that is defective at inhibiting host gene expression. It may also be desirable to switch or combine various substitutions, deletions, and insertions to further modify the phenotype of the virus. For example, the recombinant VSV background can have a deletion or mutation in the M protein.

In some embodiments other VSV proteins are modified. For instance in one embodiment the phosphoprotein (P) contains a mutation at S126. In some embodiments the mutation is a S126L mutation. In some embodiments the polymerase (L) contains a mutation at D223. In some embodiments the mutation is D223Y. In some embodiments the VSV contains mutations at both sites.

Altering the relative position of genes can also be used to attenuate virus (Clarke, et al., J. Virol., 81:2056-2064, (2007), Cooper, et al., J. Virol., 82:207-219 (2008), Flanagan, et al., J. Virol., 75:6107-6114 (2001)). VSV is highly immunogenic, and a substantial B and T cell response from the adaptive immune system will ultimately limit VSV infection, which will halt long-lasting viral infections. A virus that shows enhanced selectivity, and a faster rate of infection, will have a greater likelihood of eliminating cancer cells before the virus is eliminated by the immune system. However, the use of VSV against cancer cells does not have to be restricted to a single application. By molecular substitution of the G-protein for enhancing immune responses against foreign genes expressed by VSV, one could switch the original G protein of the virus (e.g., Indiana VSV) with the G protein from another strain or serotype (e.g., VSV New Jersey or Chandipura), allowing a slightly different antigen presentation, and reducing the initial response of the adaptive immune system to second or third oncolytic inoculations with VSV.

Therefore, the disclosed chimeric viruses can have a VSV genome that is rearranged compared to wildtype VSV. For example, shifting the L-gene to the sixth position, by rearrangement or insertion of an additional gene upstream, can result in attenuated L-protein synthesis and a slight reduction in replication (Dalton and Rose, Virology, 279(2):414-21 (2001)), an advantage when considering treatment of the brain.

Repeat passaging of virulent strains under evolutionary pressure can also be used to generate attenuated virus, increase specificity of the virus for a particular target cell type, and/or increase the oncolytic potential of the virus. For example, VSV-rp30 (“30 times repeated passaging”) is a wild-type-based VSV with an enhanced oncolytic profile (Wollmann, et al., J. Virol. 79:6005-6022 (2005)). As described in WO 2010/080909, VSV-rp30 has a preference for glioblastoma over control cells and an increased cytolytic activity on brain tumor cells. Accordingly, in some embodiments, the VSV background of the disclosed chimeric viruses is one that has been modified to attenuate the virus, increase specificity of the virus for a particular target cells, and/or increase the oncolytic potential of the virus relative to a wildtype or starting stain.

### Heterologous Glycoproteins

The disclosed chimeric VSV viruses have at least one or more heterologous glycoprotein(s). Typically, the disclosed chimeric VSV viruses are viruses that lack the G protein of VSV. Instead, the chimeric VSV viruses have one or more glycoproteins (e.g., G protein or GP protein) from a distinct, non-VSV virus or in some embodiments may have a G protein and/or F (fusion) protein from Nipah virus or other member of the Paramyxoviridae family of viruses.

Glycoproteins for a number of different viruses can be substituted into a VSV background to create a chimeric VSV that can infect cancer cells. Suitable glycoproteins can be from, for example, Lassa, rabies, lymphocytic choriomeningitis virus (LCMV), Ebola, H5N1, Nipah, Semliki Forest or Marburg virus. An Ebola-VSV chimera, and even more so a Lassa-VSV chimera or Chikungunya-VSV chimera, are particularly effective at killing brain cancers with little or no toxicity to healthy or normal cells. Other viral glycoprotein such as those from rabies, lymphocytic choriomeningitis virus (LCMV), or Marburg virus may be more suitable for targeting other cancer types, such as one or more of the cancers discussed in more detail below. It is believed that VSV chimeric viruses including an LCMV glycoprotein in place of the VSV glycoprotein may show some advantages over the VSV glycoprotein in infecting some cancer or sarcoma cells with enhanced innate immunity, such as the virus-resistant sarcoma cells described in Paglino and van den Poi, J. Virol., 85:9346-9358 (2011). In some embodiments, the G protein in the VSV chimeric virus is a heterologous G, wherein the G protein is not a G protein from LCMV. In some embodiments the chimeric VSV comprises one or more heterologous G proteins. In some embodiments the chimeric VSV comprises one or more G or F proteins.

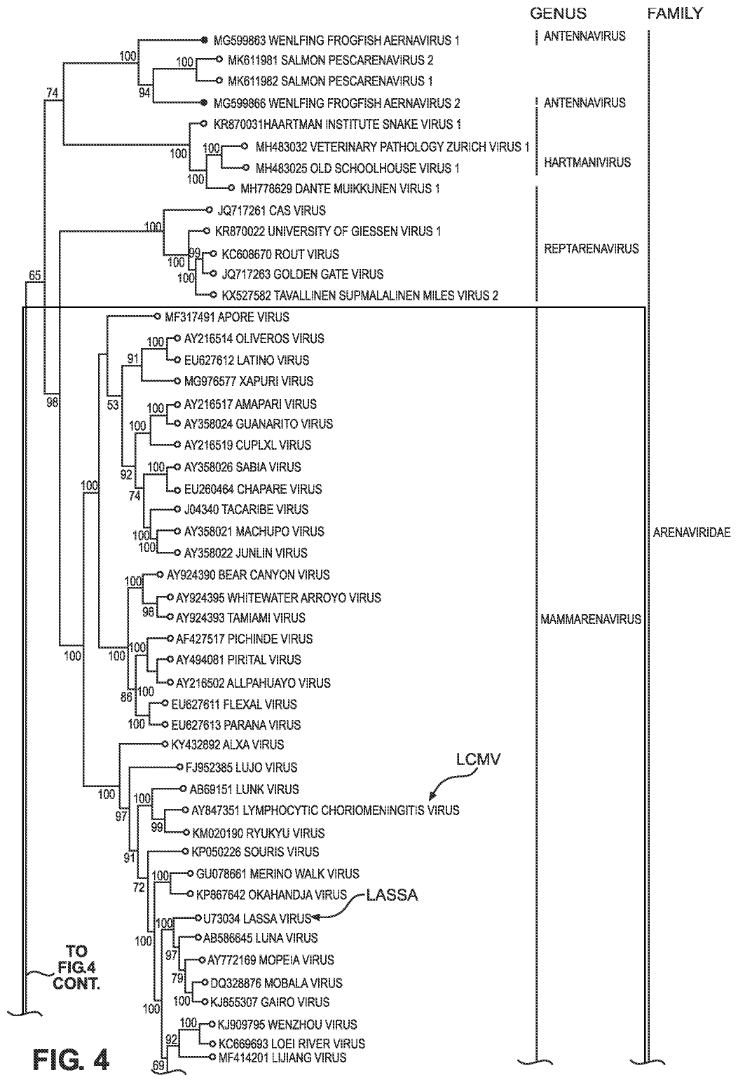

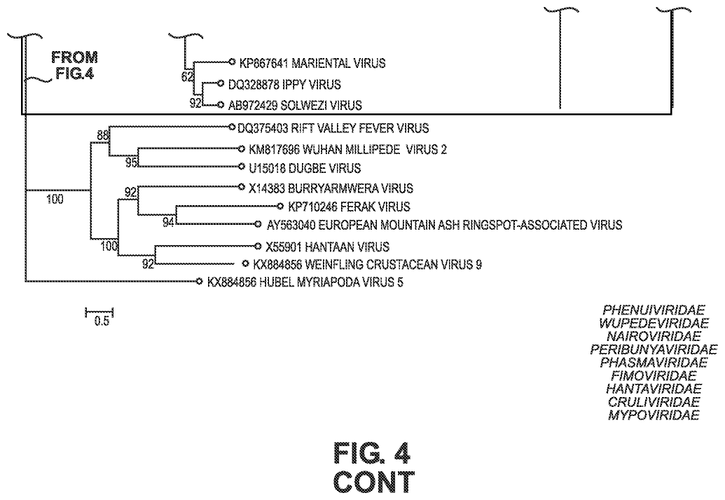

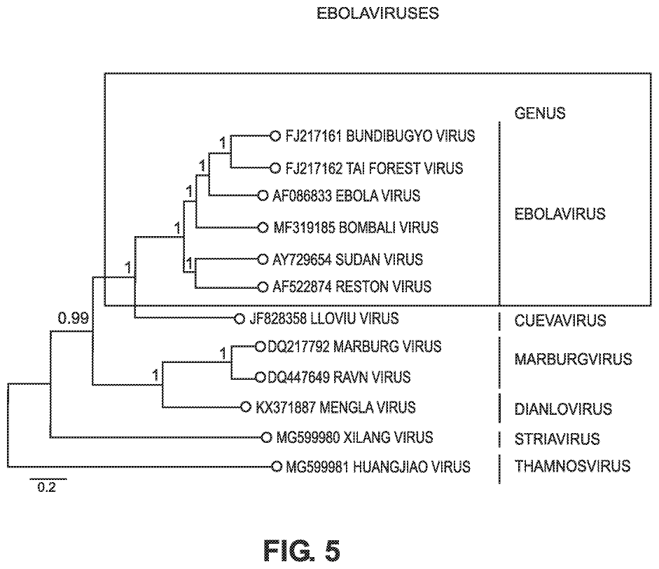

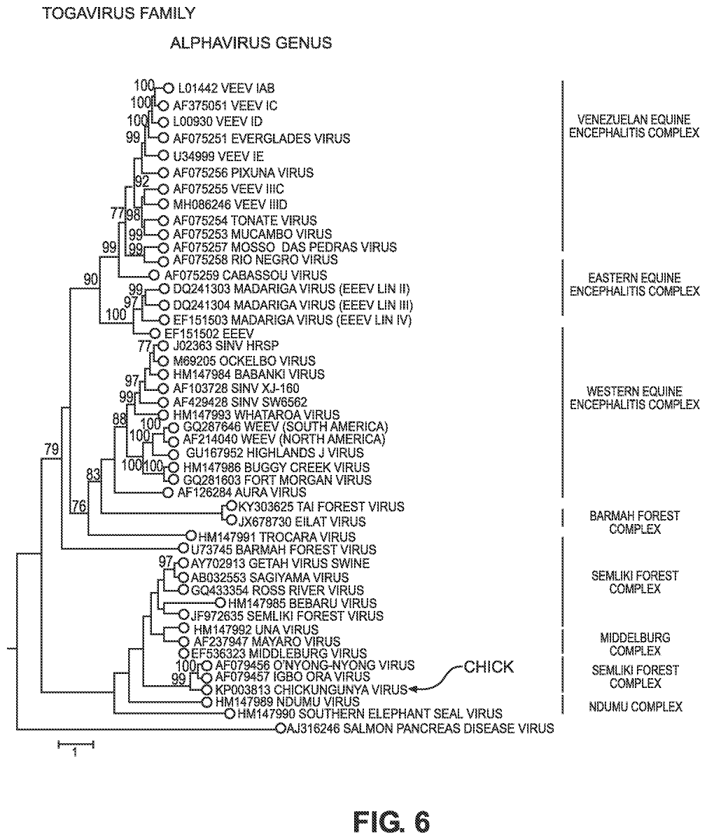

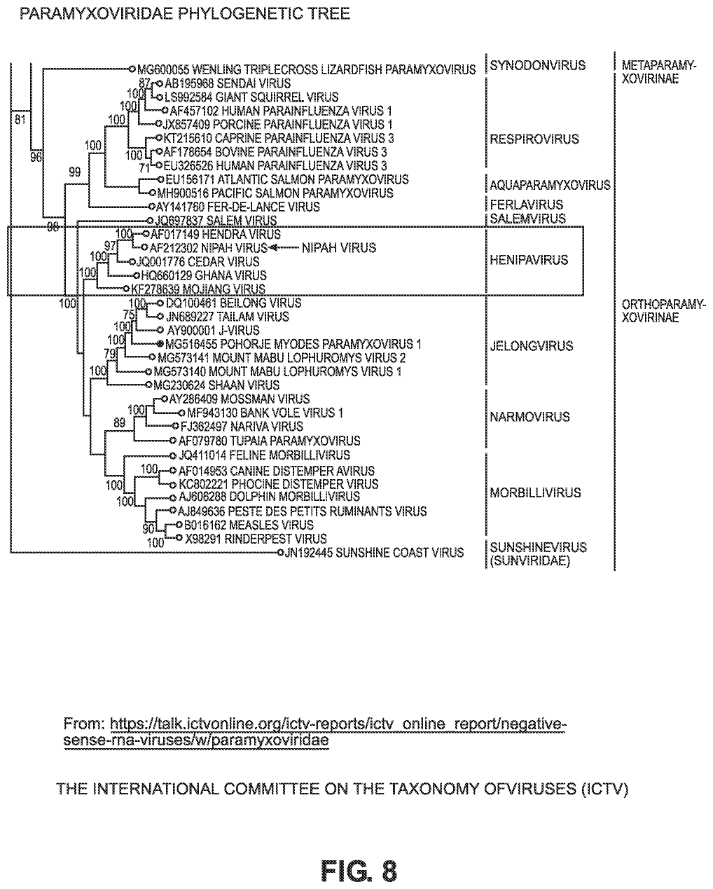

In place of the Lassa glycoprotein which has a broad spectrum of cells to which it binds, the VSV chimeric virus can have a glycoprotein from another arena virus. Other arenaviruses may have the same, similar, or different cellular binding receptors to Lassa. In some embodiments, the glycoprotein is a viral glycoprotein, preferably an arenavirus glycoprotein, that binds to one or more of the same cell receptors as Lassa glycoprotein. In some embodiments, the glycoprotein is a viral glycoprotein, preferably an arenavirus glycoprotein that binds to one or more similar cell receptors as Lassa glycoprotein. In some embodiments, the glycoprotein is an arenavirus glycoprotein that binds to different cell receptor(s) than Lassa glycoprotein. Such chimeric viruses may also be safe viruses for use in oncolysis or as vaccine vectors. Exemplary arenaviruses include, but are not limited to, Old World complex arenaviruses such as Kodoko, Lujo, Mobala, Dank, Gbagroube, Ippy, Merino Walk, Menekre, Mobala, and Mopeia, and New World arenaviruses such as Guanarito, Junin, Machupo, Sabia, Whitewater arroyo, Parana, Tamiami, Latino, plexal, and Chapare. New World arenavirus glycoproteins may target receptors different that those targeted by the Lassa glycoprotein. Additional exemplary viruses whose G proteins may find use in the VSV chimeric viruses disclosed herein are set forth in FIGS. 4, 5, 6 and 8. FIG. 4 outlines the Arenaviridae family of viruses of which Lassa virus is a member. Lassa virus is a member of the Mammarenavirus genus, which finds use in certain embodiments disclosed herein. FIG. 5 outlines the Filovirus family of which Ebola virus and Marburg virus are members. The Ebola virus is a member of the Ebolavirus genus, which finds use in certain embodiments disclosed herein. FIG. 6 outlines the Alphavirus genus of which Chikungunya is a member. Alphaviruses are members of the Togavirus Family. Alphaviruses find particular use in certain embodiments disclosed herein. FIG. 8 outlines the family Paramyxoviridae, viruses of which Nipah virus is a member. In some embodiments the G or F protein is from viruses in the Henipavirus genera, including Nipah virus, Hendra virus, Cedar virus, Ghana virus, and Mojiang virus.

### Lassa G Proteins

In the most preferred embodiment, the G protein of VSV is substituted with a glycoprotein from a Lassa virus. Lassa virus is an Arenavirus. The genomic structure or Arenaviruses and the genetic diversity of Lassa virus strains are discussed in Bowen, et al., J. Virology, 6992-7004 (2000). Viruses of the genus Arenavirus, family Arenaviridae, are enveloped viruses with a genome consisting of two single stranded RNA species designated small (S) and large (L). Each segment contains two non-overlapping genes arranged in an ambisense orientation. The viral polymerase (L protein) gene is encoded at the 3′ end of the L RNA in the genome-complementary sense, whereas the Z protein is encoded at the 5′ end of the L RNA in the genomic sense. In a similar fashion, the nucleoprotein (NP) gene is encoded at the 3′ end of the S RNA, whereas the glycoprotein precursor (GPC) is encoded at the 5′ end of the S RNA. The GPC is post-translationally cleaved into the envelope glycoproteins GP1 and GP2. The arenaviruses have been divided into two groups, the New World arenaviruses and the Old World arenaviruses. Lassa virus is an Old World arenavirus.

The glycoprotein can come from any Lassa virus. The Lassa virus glycoprotein can be from a naturally occurring virus, or a virus modified, for example, to increase or decrease the virulence of the virus, and/or increase the specificity or infectivity of the virus compared to the parental strain or serotype. Suitable strains and serotypes of Lassa virus from which the glycoprotein of the chimeric VSV virus can be derived are known in the art and include, for example, fifty-four strains identified and characterized in Bowen, et al., J. Virology, 6992-7004 (2000). Common Lassa virus stains include Lassa virus strain 803213, Lassa virus strain Acar 3080, Lassa virus strain AV, Lassa virus strain Josiah, Lassa virus strain LP, Lassa virus strain Macenta, Lassa virus strain NL, Lassa virus strain Pinneo, Lassa virus strain Weller, and Lassa virus strain Z148.

Preferably, the chimeric virus's genome, or plasmid(s) encoding the virus's genome encode the entire Lassa virus glycoprotein precursor (GPC), such that both GP1 and GP2 are expressed and contribute to formation of the chimeric virus's envelope. In some embodiments, the chimeric virus's genome, or plasmid(s) encoding the virus's genome encode less than the entire Lassa virus glycoprotein precursor (GPC). For example, in some embodiments, the viral genome or plasmid(s) encoding recombinant viral genome encodes a glycoprotein that is a truncated GPC, or only GP1 or only GP2.

The glycoprotein can be from Lassa strain Josiah. In a particular embodiment, the chimeric VSV viral genome includes the nucleic acid sequence as shown in SEQ ID NO:1. Variants can have at least 70%, 80%, 85%, 90%, 95%, 96%, 97%, 98%, 99%, or more sequence identity to SEQ ID NO:1, or to the sequence encoding an open reading frame thereof.

In one embodiment the G protein from Lassa virus strain recombinant Josiah is included (complete sequence GenBank: HQ688673.1), one or both of the open reading frames thereof, or a fragment or fragments or variants thereof encoding a functional glycoprotein.

In one embodiment the Lassa glycoprotein is encoded by a nucleic acid sequence comprising the sequence shown in SEQ ID NO:2. Variants can have at least 70%, 80%, 85%, 90%, 95%, 96%, 97%, 98%, 99%, or more sequence identity to SEQ ID NO:2, or to the sequence encoding an open reading frame thereof.

In some embodiments the Lassa glycoprotein is encoded by a nucleic acid sequence comprising the sequence shown in SEQ ID NO:3, which is a codon optimized sequence, optimized for expression in human cells. It is appreciated by those of ordinary skill in the art that when referring to a codon optimized sequence the sequence is optimized for expression in the ultimate species in which the sequence is expressed. For instance, if the sequence is to be used in mouse models, the sequence is codon optimized for expression in a mouse. If the sequence it to be used in humans, the sequence is codon optimized for expression in a human. Variants can have at least 70%, 80%, 85%, 90%, 95%, 96%, 97%, 98%, 99%, or more sequence identity to SEQ ID NO:3, or to the sequence encoding an open reading frame thereof.

In some embodiments the Lassa glycoprotein encoding nucleic acid encodes a protein having the sequence shown in SEQ ID NO: 4. That is, in some embodiments, the chimeric viral genome includes a nucleic acid sequence encoding the polypeptide as shown in SEQ ID NO:4. In some embodiments the Lassa glycoprotein comprises the sequence show in in SEQ ID NO:4 or variants thereof. Variants can encode a protein that has at least 70%, 80%, 85%, 90%, 95%, 96%, 97%, 98%, 99%, or more sequence identity to SEQ ID NO:4, or may be encoded by a nucleic acid comprising an open reading frame encoding a fragment thereof.

### Ebola G Proteins

In another preferred embodiment, the G protein of VSV is substituted with a glycoprotein from an Ebola virus. Ebola virus, along with Marburg virus, constitutes the family Filoviridae in the order of Mononegavirales (reviewed in Feldmann and Geisbert, Lancet, 377(9768): 849-862 (2011), and Sanchez, et al., Filoviridae: Marburg and Ebola viruses. In: Knipe, D M.; Howley, P M., editors. Fields virology. Philadelphia: Lippincott Williams & Wilkins; 2006. p. 1409-1448). Filoviruses are enveloped, non-segmented, negative-stranded RNA viruses with filamentous particles. Ebola virus particles have a uniform diameter of 80 nm but can greatly vary in length, with lengths up to 14000 nm. The genome includes seven genes in the order 3′ leader, nucleoprotein, virion protein (VP) 35, VP40, glycoprotein, VP30, VP24, RNA-dependent RNA polymerase (L)-5′ trailer. With the exception of the glycoprotein gene, all genes are monocistronic, encoding for one structural protein. The inner ribonucleoprotein complex of virion particles consists of the RNA genome encapsulated by the nucleoprotein, which associates with VP35, VP30, and RNA-dependent RNA polymerase to form the functional transcriptase-replicase complex. Additionally, the proteins of the ribonucleoprotein complex have other functions, for example, VP35 is an antagonist of interferon; VP40 is a matrix protein and modulates particle formation; VP24, is structural, membrane-associated protein that also interferes with interferon signaling.

The glycoprotein is the only transmembrane surface protein of the virus and forms trimeric spikes consisting of glycoprotein 1 and glycoprotein 2-two di-sulphide linked furin-cleavage fragments (Sanchez, et al., Filoviridae: Marburg and Ebola viruses. In: Knipe, D M.; Howley, P M., editors. Fields virology. Philadelphia: Lippincott Williams & Wilkins; 2006. p. 1409-1448). The primary product of the GP gene is a soluble glycoprotein (sGP) that is also secreted from infected cells, a characteristic distinguishing it from other Mononegavirales (Sanchez, et al., Proc Natl Acad Sci USA, 93:3602-3607 (1996), Volchkov, et al., Virology, 214:421-430 (1995)). Nucleic acid sequences encoding Ebola glycoprotein, the mechanism of transcription/translation yielding functional Ebola glycoprotein, Ebola glycoprotein amino acid sequences, and the structure and function of Ebola glycoprotein are well known in the art and discussed in, for example, Lee and Saphire, Future Virology, 4(6):621-635 (2009), Sanchez, Proc Natl Acad Sci USA., 93(8):3602-3607 (1996), Volchkov, et al., Virology, 214(2):421-430 (1995), Gire et al, Science, 345: 1369-1372 (2014)).

The Ebola virus glycoprotein can be from a naturally occurring virus, or a virus modified, for example, to increase or decrease the virulence of the virus, and/or increase the specificity or infectivity of the virus compared to the parental strain or serotype. Suitable species of Ebola virus from which the glycoprotein of the chimeric VSV virus can be derived are known in the art and include, for example, Sudan ebolavirus (SEBOV), Zaire ebolavirus (ZEBOV), Côte d'Ivoire ebolavirus (also known and here referred to as Ivory Coast ebolavirus (ICEBOV)), Reston ebolavirus (REBOV), and Bundigbugyo ebolavirus (BEBOV) (Geisbert and Feldmann, J. Infect. Dis., 204 (suppl 3): S1075-S1081 (2011)). Preferably, the chimeric virus's genome, or plasmid(s) encoding the virus's genome encode the entire Ebola virus glycoprotein (GP), such that the glycoprotein is expressed and contributes to formation of the chimeric virus's envelope. In some embodiments, the chimeric virus's genome, or plasmid(s) encoding the virus's genome encode less than the entire Ebola virus glycoprotein. For example, in some embodiments, the viral genome or plasmid(s) encoding recombinant viral genome encodes a glycoprotein that is a truncated or variant GP. In some embodiments, the chimeric virus's genome, or plasmid(s) encoding the virus's genome encode full length, truncated, or variant GP1, GP2, or a combination thereof.

In some embodiment, the chimeric viral genome includes the nucleic acid sequence encoding the Ebola GP SEQ ID NO:5. In some embodiments the chimeric viral genome includes the codon optimized Ebola GP encoding sequence as shown in SEQ ID NO:6. In addition, the sequence may encode a nucleic acid encoding a full-length (non-secreted) glycoprotein gene found in GenBank accession NC_002549 nt 6039-8068. In some embodiments the Ebola GP comprises the amino acid sequence shown in SEQ ID NO:7. Variants can have at least 70%, 80%, 85%, 90%, 95%, 96%, 97%, 98%, 99%, or more sequence identity to SEQ ID NO:5, 6 or 7, or to the sequence encoding an open reading frame or fragment thereof.

### Chikungunya Virus and G Protein

Chimeric viruses, particularly Chikungunya-vesicular stomatitis chimeric viruses, and compositions including an effective amount of a chimeric viruses are disclosed. The chimeric viruses are based on a VSV heterologous virus glycoproteins. At least one of the glycoproteins is typically from a Togaviridae family virus, preferably an alphavirus, most preferably a Chikungunya virus.

Alphaviruses include, but are not limited to, Eastern Equine Encephalitis virus, Venezuelan Equine Encephalitis virus, Everglades virus, Mucambo virus, Pixuna virus, Semliki Forest virus, Middelburg virus, Chikungunya virus, Onyong-Nyong virus, Ross River virus, Barmash Forest virus, Getah virus, Sagiyama virus, Berbaru virus, Mayaro virus, Una virus, Sindbis virus, Aura virus, Whataroa virus, Babanki virus, Kyzylagach virus, Western Equine Encephalitis virus, Highlands J virus, Fort Morgan virus, Ndumu virus, and Buggy Creek virus (Strauss and Strauss, Microbiological Reviews, 58(3):491-562; Weaver and Frolov, Togaviruses, p. 1010-1024. In B. W. J. Mahy and V. Meulen (ed.), Virology, vol. 2. IRL Press, Salisbury, United Kingdom; and Garmashova, et al., Journal of Virology, 81 (5) 2472-2484 (2007).

In one embodiment, the VSV G protein is supplemented or replaced with a glycoprotein from a Chikungunya virus. Chikungunya virus (CHIKV) is a positive-sense single-strand RNA virus of the alphavirus genus and Togavirus family. Prior to 2013 it was primarily found in Asia, Africa, and Europe; starting in 2013 the virus has been spread by mosquitoes through most of South America and parts of North America with non-human primates as a potential reservoir (Vignuzzi, et al., Annu. Rev. Virol., 4:181-200 (2017); Vu, et al., Clin. Lab Med., 37:371-382 (2017)). There is currently no approved vaccine available although a number of different experimental vaccines are being tested (Chattopadhyay, et al., J. Virol., 87:395-402 (2013); Powers, Clin. Microbiol. Rev., 31:e00104-16 (2018); Yang, et al., Vaccine, 35:4851-4858 (2017)). CHIKV has generally been associated with fever and joint pain, but can also cause headache, muscle ache, and rash (Hua, et al., Curr. Rheumatol. Rep., 19:69 (2017); Amdekar, et al., Virol. Immunol., 30:691-702 (2017)). The joint pain can persist for many months or longer. Chikungunya may bind to one of several prohibitin and others (Wichit, et al., Sci. Rep., 7:3145 (2017); Wintachai, et al., J. Med. Virol., 84:1757-1770 (2012)) and appears to be internalized in clathrin coated pits (Bernard, et al., PLoS One, 5:e11479 (2010); Schwartz, et al., Nat. Rev. Microbiol., 8:491-500 (2010); Hoornweg, et al., J. Virol., 90:4745-4756 (2016)).

A CHIKV-VSV chimeric virus may contain a portion of the CHIKV structural polyprotein that includes the E3-E2-6K-E1 glycoprotein sequence substituted for the VSV glycoprotein (Chattopadhyay, et al, J. Virol., 87:395-402 (2013)). CHIKV E2 underlies receptor binding, and E1 is responsible for the low pH membrane fusion activity after endocytotic entry (Voss, et al., Nature, 468:709-712 (2010); Solignat, et al., Virology, 393:183-197 (2009)). Together E2 and E1 constitute spike-like trimers on the virus surface. E3 is postulated to prevent premature virus fusion (Uchime, et al., J. Virol., 87:10255-10262 (2013)), and 6K enhances virion release and titer (Taylor, et al., J. Virol., 90:4150-4159 (2016)). VSV in which the normal glycoprotein gene G has been deleted and replaced by genes coding for the CHIKV envelope glycoprotein (VSVDG-CHIKV) has been demonstrated as safe within the brain and, as tested in rodents, did not evoke neurological dysfunction or substantive negative consequences (van den Pol, et al., J. Virol., 91:e02154-16 (2017)).

It has been discovered that recombinant, chimeric Chikungunya-VSV where the G gene is substituted with a gene encoding a Chikungunya glycoprotein protein have superior oncolytic potential in targeting and destroying cancer cells with little pathogenicity to healthy host cells.

Chikungunya VSV chimeric viruses, pharmaceutical compositions including Chikungunya VSV chimeric viruses, and methods of use thereof for treating cancer are provided. As discussed in more detail below, preferably, the virus targets and kills tumor cells, shows little or no infection of normal cells, and extended survival of tumor-bearing mice.

Most typically, the G protein of VSV is supplemented or substituted with a glycoprotein from a Chikungunya virus. In a preferred embodiment, the chimeric virus includes one or more CHIKV structural proteins (C, E3, E2, 6K and E1). In a particularly preferred embodiment, the chimeric virus includes E3, E2, 6K and E1. Chimeric virus in incorporating the entire CHIKV E3-E2-6K-E1 in place of VSV G (VSVDG-CHIKV) is in Chattopadhyay, et al., J. Virol., 87:395-402 (2013).

CHIKV structural protein and nucleic acid sequences are known in the art. See, e.g., UniProt accession no. Q8JUX5, NCBI reference sequence no. NP_690589.2, or NC_004162.2, each of which is incorporated by reference in its entirety. In one embodiment the Chikungunya GP nucleic acid is shown in SEQ ID NO:8. In one embodiment the Chikungunya GP nucleic acid is a codon optimized sequence shown in SEQ ID NO:9. In one embodiment the Chikungunya GP nucleic acid encodes a protein as shown in SEQ ID NO:10. Variants can have at least 70%, 80%, 85%, 90%, 95%, 96%, 97%, 98%, 99%, or more sequence identity to SEQ ID NO:8, 9 or 10, or to the sequence encoding an open reading frame or fragment thereof.

### Nipah G and F Proteins

The “Nipah virus” (NiV) is member of the family Paramyxoviridae, genus Henipavirus. Nipah virus is an enveloped virus with negative-stranded polarity and a non-segmented RNA genome consisting of helical nucleocapsids. Two strains of Nipah virus include, but are not limited to, the Malaysian (MY) and the Bangladesh (BD) strains. In some embodiments the heterologous G protein or proteins may actually comprise a G protein or F (fusion) protein from Nipah virus. In some embodiments, the Nipah viral envelope glycoprotein is glycoprotein F. In some embodiments, the Nipah viral envelope glycoprotein is glycoprotein G. In some embodiments, the viral vector encodes both the Nipah viral glycoprotein F and glycoprotein G. In some embodiments the Nipah G or F protein may be combined with G proteins from other viruses described herein.

In some embodiments the viral vector comprises a Nipah virus F protein having the sequence set forth in SEQ ID NO:68 and encoded by the nucleic acid sequence set forth in SEQ ID NO:67.

In some embodiments the viral vector comprises a Nipah virus G protein having the sequence set forth in SEQ ID NO:70 and encoded by the nucleic acid sequence set forth in SEQ ID NO:69.

Variants can have at least 70%, 80%, 85%, 90%, 95%, 96%, 97%, 98%, 99%, or more sequence identity to SEQ ID NO:67, 68, 69 or 70, or to the sequence encoding an open reading frame or fragment thereof.

In some embodiments the heterologous G or F protein is from a virus in the Paramyxoviridae family of viruses as shown in FIG. 8.

### Additional Transgenes

Viruses can be modified to express one or more additional transgenes, separately or as a part of other expressed proteins. The viral genome of VSV has the capacity to accommodate additional genetic material. It is thought that at least two additional transcription units, totaling 4.5 kb, can be added to the genome, and methods for doing so are known in the art. The added genes are stably maintained in the genome upon repeated passage (Schnell, et al., EMBO (1996); Schnell, et al., Journal of Virology, 70:2318-2323 (1996); Kahn, et al., Virology, 254, 81-91 (1999)).

In some embodiments the viruses are modified to include a gene encoding a therapeutic protein, an antigen, a detectable marker or reporter, a targeting moiety, or a combination thereof. In some embodiments the gene is a codon optimized gene. In some embodiments, the gene is placed in the first gene position in the VSV background. Given the nature of VSV protein expression, genes in the first position generate the highest expression of any gene in the virus, with a 3′ to 5′ decrease in gene expression. The chimeric VSV can also be constructed to contain two different and independent genes placed in the first and second gene position of VSV. For example, van den Pol and Davis, et al., J. Virol., 87(2):1019-1034 (2013), describes the generation of a highly attenuated VSV by adding two (reporter) genes to the 3′ end of the VSV genome, thereby shifting the NPMGL genes from positions 1 to 5 to positions 3 to 7. This strategy can be used to allow strong expression of genes coding for any combination of two heterologous proteins, for example two therapeutic proteins, a therapeutic protein and reporter, or an immunogenic protein and a reporter that could be useful to track the virus in a clinical situation. The chimeric VSV can also be constructed to contain two different and independent genes placed between positions 3 and 4 (i.e. between the Matrix (M) and Glycoprotein (G) coding sequenced), as well as between positions 4 and 5 (i.e. between the Glycoprotein (G) and Polymerase (L) coding sequences), although other insert locations may be used as well.

### Therapeutic Proteins and Reporters

The chimeric viruses, including Lassa, Ebola or Chikungunya VSV chimeric viruses, can be engineered to include one or more additional genes that encode a therapeutic protein or a reporter. Suitable therapeutic proteins, such as cytokines or chemokines, are known in the art, and can be selected depending on the use or disease to be treated. Preferred cytokines include, but are not limited to, granulocyte macrophage colony stimulating factor (GM-CSF), tumor necrosis factor alpha (TNFα), tumor necrosis factor beta (TNFβ), macrophage colony stimulating factor (M-CSF), interleukin-1 (IL-1), interleukin-2 (IL-2), interleukin-4 (IL-4), interleukin-5 (IL-5), interleukin-6 (IL-6), interleukin-10 (IL-10), interleukin-12 (IL-12), interleukin-15 (IL-15), interleukin-17 (IL-17), interleukin-18 (IL-18), interleukin-21 (IL-21), interferon alpha (IFNα), interferon beta (IFNβ), interferon gamma (IFNγ), and IGIF, and variants and suitable chemokines include, but are not limited to, CCL5, CCL2, CCL19, CXCL11, an alpha-chemokine or a beta-chemokine, including, but not limited to, a C5a, interleukin-8 (IL-8), monocyte chemotactic protein 1 alpha (MIP1α), monocyte chemotactic protein 1 beta (MIP1β), monocyte chemo-attractant protein 1 (MCP-1), monocyte chemo-attractant protein 3 (MCP-3), platelet activating factor (PAFR), N-formyl-methionyl-leucyl-[3H]phenylalanine (FMLPR), leukotriene B4, gastrin releasing peptide (GRP), RANTES, eotaxin, lymphotactin, IP10, I-309, ENA78, GCP-2, NAP-2, CD40L and MGSA/gro, and variants and fragments thereof. Co-stimulatory molecules include but are not limited to B7.1/CD80, 4-1BBL/CD137L, CD40L, OX-40L, GITRL, LIGHT, CD70. Checkpoint inhibitors include but are not limited to anti-CTLA4, anti-PD1 or anti PD-L1. Notable transgene combinations include but are not limited to GM-CSF plus IL-12, IL-12 plus IL-18, IL-12 plus CCL2, B7.1 plus IL-12, B7.1 plus IL-18, B7.1 plus GM-CSF, 4-1BBL plus IL-12. Other molecules include HPGD, TRIF and DAI as well as PTEN, p53, E-cad, FLT3L and CD161 and CLEC2D.

Particularly preferred genes include those that encode proteins that up-regulate an immune attack on infected tumors such as IL-28, IL-2, FLT3L, and GM-CSF (Ali, et al., Cancer Res, 65:7194-7204 (2005); Barzon, et al., Methods Mol. Biol., 542:529-549 (2009); Wongthida, et al., Hum. Gene Ther., 22:1343-53 (2011). Other therapeutic proteins that have been successfully engineered into VSV or other viruses include IL-2, IL-4, IL-7, IL-12, and TRAIL (Jinush, et al., Cancer Science, 100, 1389-1396. (2009)). Genes that find use in this disclosure include those set forth in de Graaf et al Cytokine and Growth Factor Reviews 41 (2018) 28-39, as well as Zhang and Liu, Cell Death and Disease (2020) 11:485, both of which are expressly incorporated herein by reference.

In preferred embodiments the chimeric virus expresses at least one of IL-12 or CD40L. In some embodiments the ARCH VSV comprises nucleic acids encoding one or both of IL-12 and CD40L. In addition, in some embodiments the ARCH VSV comprises nucleic acids encoding secreted and membrane-tethered IL-12 and/or CD40L. In some embodiments the ARCH VSV may comprise multiple inserts encoding either IL-12 or CD40L. In some embodiments the ARCH comprises at least one insert encoding a fusion protein that has the biological activity of a naturally occurring immunomodulatory agent. For instance, wild-type IL-12 is comprised of two subunits designated p35 and p40. As used herein, a preferred embodiment includes using a single chain fusion molecule in which the coding sequence for p35 and p40 are linked via linkers as described in U.S. Pat. No. 5,891,680, which is expressly incorporated herein by reference. That is, when the ARCH VSV comprises the fusion protein made of single chain IL-12, the IL-12 expressed is a single polypeptide with a linker joining the two parental subunits. The expressed fusion protein folds correctly and is secreted from the infected cell. Other dimeric immunomodulatory molecules may be constricted in a similar fashion and expressed as a single fusion protein with a linker separating the two parental subunits.

The polypeptide linker present in the fusion protein can be of any length and composition appropriate to join two subunits in such a manner that the resulting fusion protein has the desired biological activity and retains its integrity as a dimer or multimer. The appropriate length and composition of a linker can be determined empirically for the specific fusion protein to be produced. Generally, the polypeptide linker will be at least 10 amino acid residues. In one embodiment, the polypeptide linker is 11 to 16 amino acid residues and in specific embodiments is 11, 15 or 16 amino acid residues. In specific embodiments, the polypeptide linkers have the sequence (Gly4 Ser)3 SEQ ID NO:11; (Gly4 Ser)3 Ser SEQ ID NO:12 or (Gly4 Ser)2 Ser SEQ ID NO:13. Additional linkers may find use when expressing molecules in different species. For instance, in some embodiments, linkers are preferred when expressing molecules in human cells or mouse cells as shown herein:

(SEQ ID NO: 14)
GGGGS
Human:
(SEQ ID NO: 15)
GGTGGTGGCGGCAGT
Mouse:
(SEQ ID NO: 16)
GGAGGGGGAGGCAGC
(SEQ ID NO: 11)
(G₄S)₃
Human:
(SEQ ID NO: 17)
GGAGGCGGCGGTAGCGGAGGTGGCGGCTCCGGAGGTGGGGGGTCC
Mouse:
(SEQ ID NO: 18)
GGGGGAGGTGGCTCCGGCGGCGGCGGGTCCGGAGGTGGAGGTAGC
(SEQ ID NO: 19)
(G₄S)₂GGGLASGGS
Human:
(SEQ ID NO: 20)
GGCGGTGGTGGATCAGGAGGAGGCGGAAGCGGTGGAGGCAGTGCCTCAG
GAGGCTCT
Mouse:
(SEQ ID NO: 21)
GGAGGTGGAGGATCAGGAGGAGGGGGCTCTGGCGGCGGTAGCGCAAGTG
GCGGCTCC

These linkers can also be used to join subunits of other fusion proteins. Alternatively, other polypeptide linkers can be used to join two IL-12 subunits to produce a bioactive IL-12 fusion protein. In one embodiment the linker is encoded by the nucleic acid

(SEQ ID NO: 22)
GGTGGCGGTGGCTCGGGCGGTGGTGGGTCGGGTGGCGGCGGATCT

In one embodiment the nucleic acid encoding secreted single chain IL-12 is the sequence shown in SEQ ID NO:23 or SEQ ID NO:58 (codon optimized) and encodes the amino acid sequence of secreted IL-12 as found in (SEQ ID NO:24).

In some embodiments the IL-12 fusion protein is engineered such that it is not secreted. In this embodiment the C-terminus of the IL-12 fusion protein is modified to contain a heterologous transmembrane domain. In some embodiments it may be further modified to contain an intracellular domain. Examples of membrane anchored IL-12 molecules are found in Zhang et al. J ImmunoTherapy Cancer, 2020 and Pan et al Molecular Therapy vol. 20 no. 5, 927-937 May 2012, both of which are incorporated herein by reference.

There are a number of transmembrane domains that may be fused to the C-terminus of the IL-12 fusion protein. In some embodiments the fusion protein comprises a linker between the IL-12 fusion protein and the transmembrane domain. Exemplary transmembrane domains that may be used include the transmembrane domain from any transmembrane protein. Preferred transmembrane proteins from which a transmembrane domain may be used include receptors, such as growth factor receptor receptors, tyrosine kinase receptors, transmembrane guanylyl cyclase receptors, cytokine receptors and the like. In some embodiments the transmembrane domain is from CD8, CD28 or B7.1 In one embodiment the transmembrane is the B7-1 transmembrane domain. Preferred mouse and human B7-1 transmembrane domains comprise the amino acid and nucleic acid sequence shown below

### Transmembrane Domains

B7.1 (human, codon-optimized)
(SEQ ID NO: 25)
GACAATTTGTTGCCCAGTTGGGCGATTACCCTTATATCAGTTAATGGCA
TATTTGTTATTTGCTGTCTCACTTACTGCTTCGCCCCAAGGTGCCGCGA
GAGACGCCGAAATGAAAGGCTGAGGCGAGAGTCAGTGCGGCCAGTC
Human Amino acid:
(SEQ ID NO: 26)
DNLLPSWAITLISVNGIFVICCLTYCFAPRCRERRRNERLRRESVRPV
B7.1 (mouse, codon-optimized)
(SEQ ID NO: 27)
CCCCCTGAAGACCCTCCAGACAGTAAGAATACACTTGTGTTGTTTGGCG
CTGGATTCGGAGCAGTAATAACCGTTGTAGTTATTGTGGTTATCATTAA
ATGTTTTTGTAAGCATCGCTCCTGTTTTAGAAGGAACGAGGCATCCCGA
GAAACTAATAATAGTTTGACATTCGGACCCGAGGAGGCATTGGCAGAAC
AGACAGTCTTTCTC
Mouse Amino acid:
(SEQ ID NO: 28)
PPEDPPDSKNTLVLFGAGFGAVITVVVIVVIIKCFCKHRSCFRRNEASR
ETNNSLTFGPEEALAEQTVFL
CD28 (human, codon-optimized)
(SEQ ID NO: 29)
GTCCTCGTTGTCGTGGGTGGTGTACTGGCTTGTTATTCACTTCTTGTAA
CCGTTGCGTTCATAATTTTC
Human Amino acid:
(SEQ ID NO: 30)
VLVVVGGVLACYSLLVTVAFIIF
CD28 (mouse, codon-optimized)
(SEQ ID NO: 31)
TTTTGGGCACTCGTAGTAGTTGCTGGTGTGTTGTTTTGCTACGGACTGC
TCGTAACTGTCGCACTTTGCGTCATTTGGACT
Mouse Amino acid:
(SEQ ID NO: 32)
FWALVVVAGVLFCYGLLVTVALCVIWT
CD8 (human, codon-optimized)
(SEQ ID NO: 33)
ATATACATCTGGGCCCCACTCGCCGGAACGTGCGGCGTCTTGCTTCTCA
GCCTGGTGATCACA
Human Amino acid:
(SEQ ID NO: 34)
IYIWAPLAGTCGVLLLSLVIT
CD8 (mouse, codon-optimized)
(SEQ ID NO: 35)
ATATGGGCACCCCTGGCTGGCATTTGTGTTGCATTGCTTTTGTCACTGA
TAATTACCTTGATT
Mouse Amino acid:
(SEQ ID NO: 36)
IWAPLAGICVALLLSLIITLI

In one embodiment the amino acid sequence of membrane anchored IL-12 is found in SEQ ID NO:38 and is encoded by the sequence as shown in SEQ ID NO:37 or SEQ ID NO:59 (codon optimized).

Nucleic acids encoding mouse secreted single chain IL-12 are found in SEQ ID NO:39 and 65 (codon optimized) and encode a mouse single chain IL-12 having the sequence shown in SEQ ID NO:40. Nucleic acids encoding mouse membrane anchored single chain IL-12 are found in SEQ ID NO:41 and 60 (codon optimized) and encode mouse membrane anchored IL-12 having the sequence shown in SEQ ID NO:42.

In some embodiments the ARCH VSV comprises a nucleic acid encoding CD40L, which may be secreted. Nucleic acids encoding secreted CD40L comprises the sequence shown in SEQ ID NO: 43 and 61 (codon optimized) and encodes the amino acid sequence shown in SEQ ID NO: 44.

In some embodiments the ARCH VSV comprises a nucleic acid encoding membrane anchored CD40L. In this embodiment, the C-terminus of the CD40L is modified to contain a heterologous transmembrane domain. In some embodiments it may be further modified to contain an intracellular domain. In an alternative embodiment CD40L sequence is modified such that the metalloproteinase cleavage site ¹¹⁰SFEMQKG¹¹⁶ (SEQ ID NO:66) is deleted (sequences locations in superscript are relative to the sequence shown in SEQ ID NO:44. Membrane anchored CD40L, which is lacking a metalloproteinase cleavage site is encoded by the nucleic acid shown in SEQ ID NO: 45 or 62 (codon optimized). This encodes the amino acid sequence shown in SEQ ID NO:46. The nucleic acids deleted from the nucleic acid encoding the secreted CD40L are found in SEQ ID NO:47.

In some embodiments the ARCH VSV comprises a nucleic acid encoding mouse CD40L, which may be secreted. Secreted mouse CD40L comprises the sequence shown in SEQ ID NO: 48 or 63 (codon optimized) and encodes the amino acid sequence shown in SEQ ID NO:49.

In some embodiments the ARCH VSV comprises a nucleic acid encoding mouse membrane anchored CD40L. In this embodiment, the C-terminus of the mouse CD40L is modified to contain a heterologous transmembrane domain. In some embodiments it may be further modified to contain an intracellular domain. In an alternative embodiment CD40L sequence is modified such that the metalloproteinase cleavage site is deleted. Membrane anchored mouse CD40L, which is lacking a metalloproteinase cleavage site is encoded by the nucleic acid shown in SEQ ID NO: 50 or 64 (codon optimized). This encodes the amino acid sequence shown in SEQ ID NO:51. The nucleic acids deleted from the nucleic acid encoding the secreted CD40L are found in SEQ ID NO:52.

In view of the above, in some embodiments a preferred virus of the disclosure comprises the sequence shown in SEQ ID NO:53, which encodes a chimeric VSV, comprising a Lassa G protein and secreted IL-12. In this sequence the nucleic acids encoding secreted IL-12 are found between positions 4 and 5. In one embodiment a preferred virus of the disclosure comprises the sequence shown in SEQ ID NO:54, which encodes a chimeric VSV, comprising a Lassa G protein and CD40L. In this sequence the nucleic acids encoding CD40L are found between positions 4 and 5.

In view of the above, in some embodiments a preferred virus of the disclosure comprises the sequence shown in SEQ ID NO:55, which encodes a chimeric VSV, comprising a Lassa G protein and single chain, transmembrane IL-12. In this embodiment, the transmembrane domain is the B7.1 transmembrane domain although others may be used as described herein. In this embodiment the nucleic acid sequence is codon optimized.

In one embodiment a preferred virus of the disclosure comprises the sequence shown in SEQ ID NO:56, which encodes a chimeric VSV, comprising a Lassa G protein and membrane anchored CD40L. In this embodiment the nucleic acid sequence is codon optimized.

In one embodiment a preferred virus of the disclosure comprises the sequence shown in SEQ ID NO:57, which encodes a chimeric VSV, comprising a Lassa G protein and single chain, membrane anchored IL-12 and membrane anchored CD40L. In this embodiment the transmembrane domain in the membrane anchored IL-12 is from B7.1, although other transmembrane domains could be used as described herein. In this embodiment the nucleic acid sequence is codon optimized. In one embodiment the virus of the disclosure comprises the sequence shown in SEQ ID NO:71, which encodes a chimeric VSV comprising Lassa G protein and murine codon optimized CD40L and IL12. In one embodiment the virus of the disclosure comprises the sequence shown in SEQ ID NO:78, which encodes chimeric VSV full-length sequence that consists of the VSV backbone from FJ478454 containing the chimeric cassette in place of VSV G. This cassette consists of mammalian codon optimized Lassa G, human CD40L and human IL12.

As noted previously, in some embodiments the ARCH VSV expresses both secreted and transmembrane immunomodulatory molecules. For instance, the ARCH VSV may express both membrane anchored and secreted IL-12 or CD40L. In some embodiments the ARCH VSV expresses secreted IL-12 and membrane anchored CD40L. In some embodiments the ARCH VSV expresses secreted CD40L and membrane anchored IL-12. In some embodiments ARCH VSV expresses two or more secreted IL-12 molecules, two or transmembrane IL-12 molecules, two or more secreted CD40L molecules, and/or two or more membrane anchored CD40L molecules.

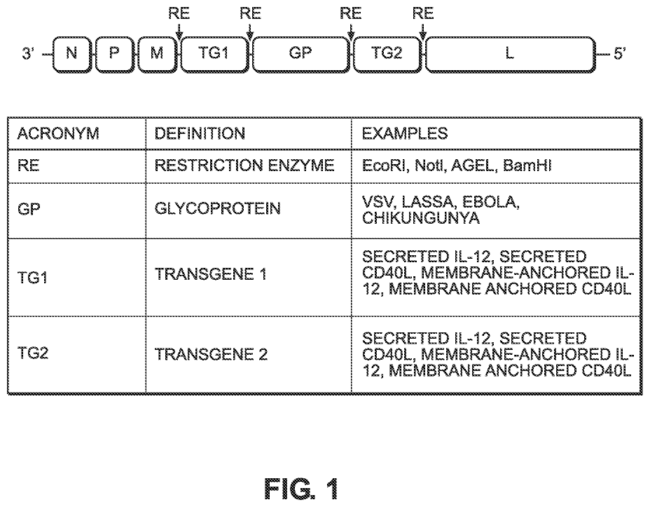

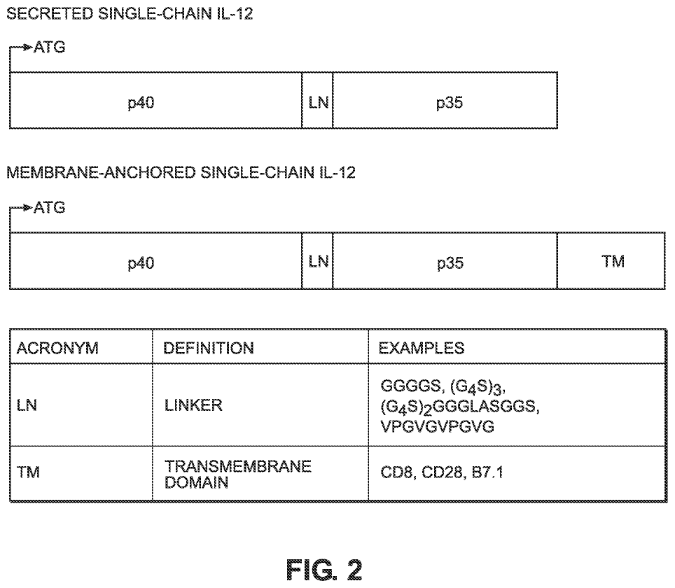

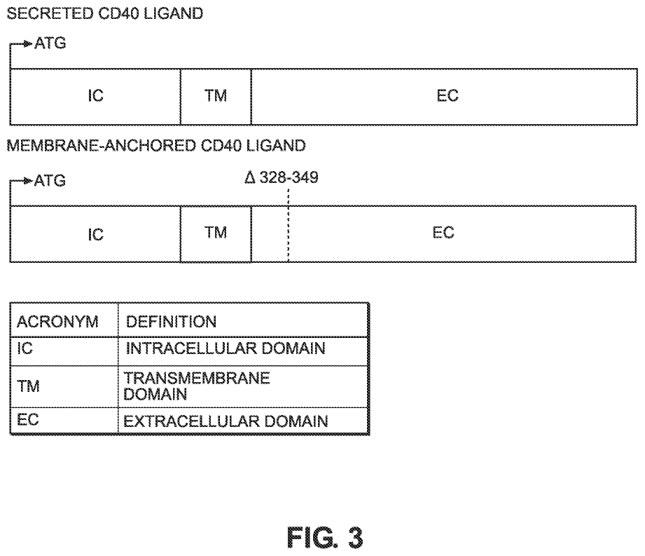

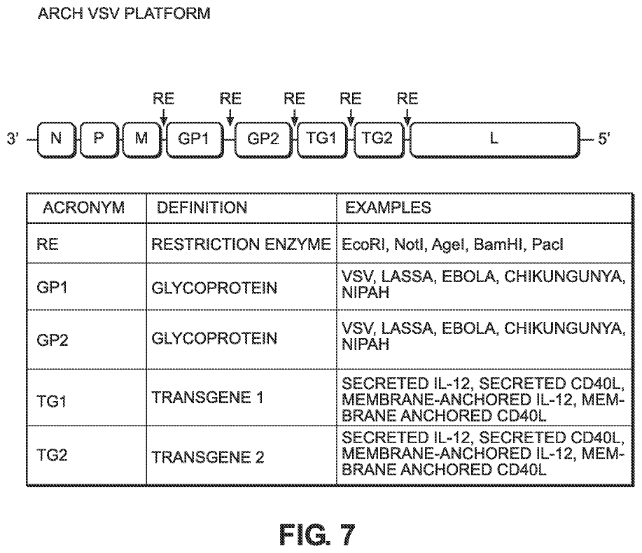

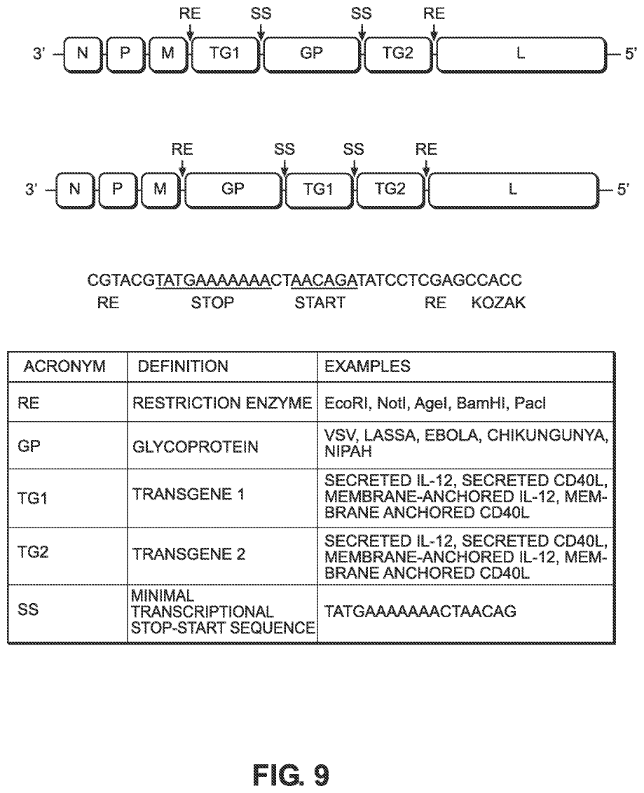

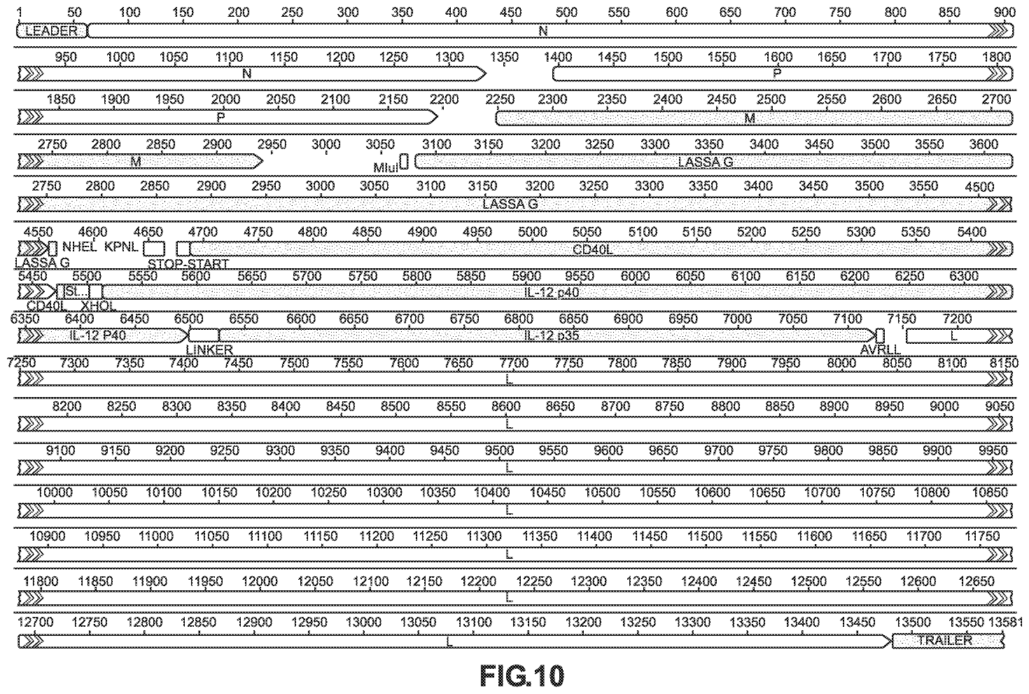

A benefit of the ARCH VSV platform as described herein is the power of the flexibility of the system in which viruses can be customized with different immunomodulatory molecules and/or heterologous G proteins. As shown in FIG. 1 the combinatorial possibilities of using two different immunomodulatory molecules with different G proteins creates a system to tailor a virus for different purposes. Moreover, the ARCH VSV platform can comprise on or more G proteins or in some embodiments may comprise a G and/or F protein (FIG. 7) or may include transgenes in different configurations with respect to the G protein as shown in FIG. 9. For instance, in some embodiments a first transgene is 5′ of the G protein while a second transgene is 3′ of the G protein. In some embodiments, the transgenes are both on the 5′ side of the G protein and in some embodiments both of the transgenes are on the 3′ side of the G protein.

In some embodiments, the endogenous VSV G protein is replaced with a heterologous cassette. By “heterologous cassette” is meant nucleic acid encoding one or multiple heterologous sequences that is inserted into the VSV genome in place of the coding sequence of the endogenous VSV G protein. The heterologous cassette may encode a heterologous G and/or F protein and one or more immunomodulatory molecules. In some embodiments the chimeric cassette may be flanked with restriction endonuclease sites. These sites may be any sites recognized by any restriction enzyme but preferred restriction enzymes are EcoR1, Not1, Age1 and BamH1.

In some embodiments the chimeric cassettes contain stop-start sequences between the coding sequences for the different proteins. For instance there may be a minimal transcriptional stop-start sequence between the transgenes and G proteins as shown in FIG. 9. A minimal transcriptional start-stop sequence is shown in SEQ ID NO: 75. A start-stop sequence containing restriction sites and a Kozak sequence is found in SEQ ID NO:76.

Viruses can be modified to express one or more additional transgenes, separately or as a part of other expressed proteins. The viral genome of VSV has the capacity to accommodate additional genetic material. At least two additional transcription units, totaling 4.5 kb, can be added to the genome, and methods for doing so are known in the art. The added genes are stably maintained in the genome upon repeated passage (Schnell, et al., EMBO (1996); Schnell, et al., Journal of Virology, 70:2318-2323 (1996); Kahn, et al., Virology, 254, 81-91 (1999)).

In some embodiments the viruses are modified to include a gene encoding a therapeutic protein, an antigen, a detectable marker or reporter, a targeting moiety, or a combination thereof. In some embodiments, the gene is placed in the first gene position in the VSV background. Given the nature of VSV protein expression, genes in the first position generate the highest expression of any gene in the virus, with a 3′ to 5′ decrease in gene expression. The chimeric VSV can also be constructed to contain two different and independent genes placed in the first and second gene position of VSV. For example, van den Pol and Davis, et al., J. Virol., 87(2):1019-1034 (2013), describes the generation of a highly attenuated VSV by adding two (reporter) genes to the 3′ end of the VSV genome, thereby shifting the NPMGL genes from positions 1 to 5 to positions 3 to 7. This strategy can be used to allow strong expression of genes coding for any combination of two heterologous proteins, for example two therapeutic proteins, a therapeutic protein and reporter, or an immunogenic protein and a reporter that could be useful to track the virus in a clinical situation.

The chimeric virus can be further modified to express one or more therapeutic proteins, reporters, vaccine antigens, or targeting moieties. The chimeric viruses can be replication competent or incompetent. The chimeric viruses can be included in a pharmaceutical formulation alone or in combination with other therapeutic agents an effective amount of the virus to reduce one or more symptoms of cancer.

Accordingly, in some embodiments preferred configurations of the heterologous cassette include from one to 10 heterologous coding sequences, preferably 2-5 heterologous coding sequences, preferably 3-4 heterologous coding sequences. The heterologous coding sequences may encode heterologous G proteins as described herein, heterologous F proteins as described herein and/or immunomodulatory molecules as described herein. That is, the disclosure provides multiple different configurations of the heterologous coding sequences in the chimeric cassette. For example, the cassette may be configured with G-TG1-TG2, G-TG2-TG1, TG1-G-TG2, TG1-TG2-G, TG2-G-TG1, TG2-TG2-G, wherein G refers to heterologous G protein, TG1 refers to first transgene and TG2 refers to second transgene. Referring to specific transgenes, the configurations include G-CD-IL, G-IL-CD, CD-G-IL, CD-IL-G, IL-G-CD, IL-CD-G, wherein G refers to heterologous G protein, CD refers to CD40L as described herein and IL refers to IL-12, as described herein.

Once made, the chimeric oncolytic viruses described herein find use in treating cancers which may also be described as “neoplastic cells,” “neoplasia,” “tumor,” “tumor cells,” “cancer” and “cancer cells,” (used interchangeably), all of which refer to cells which exhibit relatively autonomous growth, so that they exhibit an aberrant growth phenotype characterized by a significant loss of control of cell proliferation (i.e., de-regulated cell division). Neoplastic cells can be malignant or benign. In some embodiments the cancer may be a glioma, glioblastoma, ovarian cancer, breast cancer, melanoma. In some embodiments the cancer which may be treated may be Acute granulocytic leukemia. Acute lymphocytic leukemia. Acute myelogenous leukemia, Adenocarcinoma, Adenosarcoma, Adrenal cancer. Adrenocortical carcinoma, Anal cancer. Anaplastic astrocytoma, Angiosarcoma, Appendix cancer, Astrocytoma. Basal cell carcinoma. B-Cell lymphoma). Bile duct cancer. Bladder cancer, Bone cancer, Bowel cancer, Brain cancer, Brain stem glioma, Brain tumor, Breast cancer. Carcinoid tumors. Cervical cancer, Cholangiocarcinoma. Chondrosarcoma, Chronic lymphocytic leukemia. Chronic myelogenous leukemia. Colon cancer, Colorectal cancer, Craniopharyngioma, Cutaneous lymphoma. Cutaneous melanoma, Diffuse astrocytoma, Ductal carcinoma in situ. Endometrial cancer, Ependymoma, Epithelioid sarcoma, Esophageal cancer, Ewing sarcoma, Extrahepatic bile duct cancer, Eye cancer, Fallopian tube cancer, Fibrosarcoma. Gallbladder cancer, Gastric cancer, Gastrointestinal cancer. Gastrointestinal carcinoid cancer, Gastrointestinal stromal tumors, General, Germ cell tumor, Glioblastoma multiforme, Glioma, Hairy cell leukemia. Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer. Invasive/infiltrating breast cancer, Islet cell cancer, Jaw cancer, Kaposi sarcoma. Kidney cancer. Laryngeal cancer, Leiomyosarcoma, Leptomeningeal metastases, Leukemia, Lip cancer, Liposarcoma. Liver cancer, Lobular carcinoma in situ. Low-grade astrocytoma. Lung cancer. Lymph node cancer, Lymphoma. Male breast cancer, Medullary carcinoma. Medulloblastoma. Melanoma, Meningioma, Merkel cell carcinoma, Mesenchymal chondrosarcoma. Mesenchymous, Mesothelioma, Metastatic breast cancer. Metastatic melanoma, Metastatic squamous neck cancer, Mixed gliomas, Mouth cancer, Mucinous carcinoma. Mucosal melanoma. Multiple myeloma. Nasal cavity cancer, Nasopharyngeal cancer. Neck cancer, Neuroblastoma. Neuroendocrine tumors, Non-Hodgkin lymphoma. Non-Hodgkin's lymphoma, Non-small cell lung cancer. Oat cell cancer. Ocular cancer, Ocular melanoma, Oligodendroglioma, Oral cancer, Oral cavity cancer, Oropharyngeal cancer, Osteogenic sarcoma, Osteosarcoma, Ovarian cancer. Ovarian epithelial cancer, Ovarian germ cell tumor. Ovarian primary peritoneal carcinoma, Ovarian sex cord stromal tumor, Paget's disease, Pancreatic cancer, Papillary carcinoma. Paranasal sinus cancer, Parathyroid cancer, Pelvic cancer, Penile cancer. Peripheral nerve cancer, Peritoneal cancer. Pharyngeal cancer, Pheochromocytoma. Pilocytic astrocytoma, Pineal region tumor. Pineoblastoma, Pituitary gland cancer, Primary central nervous system lymphoma, Prostate cancer, Rectal cancer. Renal cell cancer, Renal pelvis cancer, Rhabdomyosarcoma. Salivary gland cancer. Sarcoma, Sarcoma, bone, Sarcoma, soft tissue, Sarcoma, uterine. Sinus cancer, Skin cancer, Small cell lung cancer, Small intestine cancer, Soft tissue sarcoma, Spinal cancer. Spinal column cancer. Spinal cord cancer, Spinal tumor, Squamous cell carcinoma. Stomach cancer, Synovial sarcoma. T-cell lymphoma), Testicular cancer. Throat cancer, Thymoma/thymic carcinoma, Thyroid cancer. Tongue cancer. Tonsil cancer, Transitional cell cancer. Transitional cell cancer. Transitional cell cancer, Triple-negative breast cancer. Tubal cancer, Tubular carcinoma. Ureteral cancer. Ureteral cancer, Urethral cancer, Uterine adenocarcinoma, Uterine cancer. Uterine sarcoma, Vaginal cancer, and Vulvar cancer. In some embodiments the cancer to be treated is metastatic cancer.

The chimeric oncolytic virus of the present invention may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the mvocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

In some embodiments, compositions may be administered in a way which allows them to cross the blood-brain barrier, vascular barrier, or other epithelial barrier. The chimeric oncolytic virus of the present invention may be administered in any suitable form, either as a liquid solution or suspension, as a solid form suitable for liquid solution or suspension in a liquid solution. The chimeric oncolytic virus may be formulated with any appropriate and pharmaceutically acceptable excipient.

In one embodiment, the chimeric oncolytic virus of the present invention may be delivered to a subject via a single route administration.

In one embodiment, the chimeric oncolytic virus of the present invention may be delivered to a subject via a multi-site route of administration. A subject may be administered at 2, 3, 4, 5, or more than 5 sites.

In one embodiment, a subject may be administered the chimeric oncolytic virus of the present invention using a bolus infusion.

In one embodiment, a subject may be administered the chimeric oncolytic virus of the present invention using sustained delivery over a period of minutes, hours, or days. The infusion rate may be changed depending on the subject, distribution, formulation or another delivery parameter.

In one embodiment, the chimeric oncolytic virus of the present invention may be delivered by intramuscular delivery route. Non-limiting examples of intramuscular administration include an intravenous injection or a subcutaneous injection.

In one embodiment, the chimeric oncolytic virus of the present invention may be delivered by oral administration. Non-limiting examples of oral administration include a digestive tract administration and a buccal administration.

In one embodiment, the chimeric oncolytic virus of the present invention may be delivered by intraocular delivery route A non-limiting example of intraocular administration include an intravitreal injection.

In one embodiment, the chimeric oncolytic virus of the present invention may be delivered by intranasal delivery route. Non-limiting examples of intranasal delivery include administration of nasal drops or nasal sprays.

In some embodiments, the chimeric oncolytic virus that may be administered to a subject by peripheral injections. Non-limiting examples of peripheral injections include intraperitoneal, intramuscular, intravenous, conjunctival, or joint injection. In one embodiment, the chimeric oncolytic virus may be delivered by injection into the CSF pathway. Non-limiting examples of delivery to the CSF pathway include intrathecal and intracerebroventricular administration.

In one embodiment, the chimeric oncolytic virus may be delivered by systemic delivery. As a non-limiting example, the systemic delivery may be by intravascular administration.

In one embodiment, the chimeric oncolytic virus of the present invention may be administered to a subject by intracranial delivery.

In one embodiment, the chimeric oncolytic virus of the present invention may be administered to a subject by intraparenchymal administration.

In one embodiment, the chimeric oncolytic virus of the present invention may be administered to a subject by intramuscular administration.

In one embodiment, the chimeric oncolytic virus of the present invention may be administered to a subject by intravenous administration.

In one embodiment, the chimeric oncolytic virus of the present invention may be administered to a subject by subcutaneous administration.

In one embodiment, the chimeric oncolytic virus of the present invention may be administered to a subject by topical administration.

In one embodiment, the chimeric oncolytic virus may be delivered by direct injection into the brain. As a non-limiting example, the brain delivery may be by intrastriatal administration.

In one embodiment, the chimeric oncolytic virus may be delivered by more than one route of administration. As non-limiting examples of combination administrations, chimeric oncolytic virus may be delivered by intrathecal and intracerebroventricular, or by intravenous and intraparenchymal administration.

While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the present invention.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Expression and Localization of the Heterologous Immunomodulatory Molecules

Replication-competent ARCH VSV expressing one or more immunomodulatory transgenes is produced using a reverse genetics system. This system consists of five plasmids expressing: 1) a complete ARCH genome containing one or more immunomodulatory genes; 2) the wild-type VSV nucleocapsid (N); 3) the wild-type VSV phosphoprotein (P); 4) the wild-type VSV polymerase; and 5) a T7 polymerase. These plasmids are transfected into Vero cells to produce infectious viral particles. The viral particles are harvested, concentrated by centrifugation, and filtered. These viral particles are used to infect various human and mouse glioma cell lines, including U87 and CT2A. After 24 hours, the cells (i.e., cell lysate) and their culture media are harvested. Protein expression levels of the immunomodulatory molecules are determined using Western Blotting. Localization of the immunomodulatory molecules are determined by assessing whether immunomodulatory proteins are detected in the cell lysate or cell culture media. Secreted forms of the immunomodulatory molecules will appear in the cell lysate and cell culture media. Membrane-anchored forms of the immunomodulatory molecules will only be present in the cell lysates.

### Example 2

Improved Survival in Syngeneic Mouse Glioma Models Treated with Arch VSV

Mouse glioma CT-2A cells expressing luciferase (Sigma-Aldrich) are used to establish unilateral intracranial transplants in 6- to 8-week-old female C57BL/6 mice by injection of 5×104 cells into the right striatum. Approximately two weeks after tumor placement, mice in the treatment cohorts (n=8) receive an intratumoral injection of recombinant virus (3.6×104 PFU). Control groups are treated with either saline, wild-type VSV or unarmed chimeric VSV (i.e. parental viruses). Experimental groups are treated with ARCH VSV. Overall survival of mice improves when treated by intratumoral injection of ARCH VSV relative to all control mice, including those treated with unarmed chimeric VSV.

### Example 3

### Enhanced Immunologic Memory Responses to Gliomas Mediated by Arch VSV

Tumor rechallenge experiments assess the immunologic memory responses induced by immunomodulatory transgenes by injecting CT-2A cells into the contralateral hemisphere in a cohort of previously treated mice that successfully survived >90 days. A similar tumor cell inoculum is administered to treatment-naïve mice as a control. Rechallenged mice are followed for at least 60 additional days and receive no additional therapy. Successfully induced immunological memory, mediated by the expression of immunomodulatory transgense, results in the re-challenged mice surviving longer than those treated with parental control viruses.

### Example 4

Treatment of Mouse Glioma Models with Arch VSV Enhances T Cell Infiltration into the Tumor

To assess the effects of ARCH VSV treatment on the tumor microenvironment and host immune activation, CT-2A gliomas are resected several days post ARCH VSV treatment. Immunohistochemical analysis and flow cytometry is used to assess the extent of CD8+ and CD4+ T cell infiltration into the tumor. The extent of tumor infiltrating lymphocytes specific for VSV is compared to the frequency of VSV-specific lymphocytes in the spleen of the same animal. Higher frequencies of VSV-specific lymphocytes in the tumor suggest T cells are enriched in the tumor microenvironment. Comparisons between cohorts of animals treated with ARCH VSV and parental control viruses suggests the extent to which immunomodulatory transgenes can stimulate tumor-targeted T cell infiltration.

1. An armed oncolytic virus comprising a chimeric Vesicular Stomatitis Virus (VSV), wherein the chimeric VSV comprises:
a. a VSV background and at least one heterologous viral glycoprotein selected from the group consisting of an Arenaviridae family virus, a Filovirus family virus, Paramyxoviridae family virus and a Togovirus family virus; and
b. at least one heterologous immunomodulatory molecule.

2. The armed oncolytic virus according to claim 1, wherein said heterologous glycoprotein is selected from the group consisting of Lassa virus glycoprotein, an Ebola virus glycoprotein, a Chikungunya virus glycoprotein, and functional fragment or fragments thereof, in place of the VSV G-protein.

3. The armed oncolytic virus according to claim 1, wherein the immunomodulatory molecule is selected from the group consisting of secreted single chain IL-12, membrane anchored IL-12, secreted CD40L and membrane anchored CD40L.

4. The armed oncolytic virus according to claim 2, wherein the secreted single chain and membrane anchored IL-12 are a fusion protein of p35 and p40 subunits fused by a linker.

5. The armed oncolytic virus according to claim 3, wherein said membrane anchored IL-12 comprises a transmembrane domain.

6. The armed oncolytic virus according to claim 4, wherein the transmembrane is selected from the group consisting of CD28, CD8 and B7.1 transmembrane domains.

7. The armed oncolytic virus according to claim 3, wherein said membrane anchored CD40L lacks the endogenous metalloprotease cleavage site.

8. The armed oncolytic virus according to claim 1, wherein the heterologous immunodulatory molecule is not wild-type IL-12.

9. The armed oncolytic virus according to claim 1 wherein the sequences are codon optimized for expression in human cells.

10. A nucleic acid encoding the armed oncolytic virus according to claim 1.

11. A nucleic acid encoding an armed oncolytic virus comprising a nucleic acid encoding a chimeric VSV virus wherein the coding sequence for the endogenous G protein is replaced with a chimeric cassette comprising a nucleic acid encoding a first and second transgene comprising a first and second immunomodulatory molecule and a heterologous viral glycoprotein selected from the group consisting of an Arenaviridae family virus, a Filovirus family virus, Paramyxoviridae family virus and a Togovirus family virus.

12. The nucleic acid according to claim 11 wherein said chimeric cassette comprises from 3′ to 5′ a first coding sequence, a second coding sequence and a third coding sequence.

13. The nucleic acid according to claim 12 further comprising a first minimal transcriptional stop-start sequence between said first coding sequence and said second coding sequence and a second minimal transcriptional stop-start sequence between said second coding sequence and said third coding sequence.

14. The nucleic acid according to claim 13, wherein at least one of said minimal transcriptional stop-start sequences comprises SEQ ID NO:75.

15. The nucleic acid according of claim 11, wherein the nucleic acid encoding said heterologous glycoprotein encodes a protein selected from the group consisting of Lassa virus glycoprotein, an Ebola virus glycoprotein, a Chikungunya virus glycoprotein, and functional fragment or fragments thereof, in place of the VSV G-protein.

16. The nucleic acid according to claim 11, wherein the nucleic acid encoding said immunomodulatory molecule encodes a protein selected from the group consisting of secreted single chain IL-12, membrane anchored IL-12, secreted CD40L and membrane anchored CD40L.

17. A method of treating cancer in a subject in need thereof comprising administering to said subject an armed oncolytic virus as described in any of claims 1-7.
